(19) United States
(12) Patent Application Publication
(10) Pub. No.: US 2023/0104609 A1
(43) Pub. Date: Apr. 6, 2023

(54) DIAGNOSTIC SYSTEM AND METHODS OF USING AND MANUFACTURING THE SAME

(71) Applicants: TINTORIA PIANA, INC, Cartersville, GA (US); Consiglio Nazionale delle Ricerche (CNR), Rome (IT); Università Magna Graecia (UMG), Catanzaro (IT)

(72) Inventors: Andrea Piana, Cartersville, GA (US); Michael Stephen DeFranks, Cartersville, GA (US); Sang-hoon Lim, Cartersville, GA (US); Andy Hollis, Cartersville, GA (US); Matteo Cocuzza, Torino (IT); Valentina Sinisi, Parma (IT); Andrea Zappettini, Reggio Emilia (IT); Natalia Malara, Catanzaro (IT); Marialaura Coluccio, Catanzaro (IT)

(21) Appl. No.: 17/958,486

(22) Filed: Oct. 3, 2022

Related U.S. Application Data
(60) Provisional application No. 63/253,635, filed on Oct. 8, 2021.
(60) Provisional application No. 63/251,734, filed on Oct. 4, 2021.

(51) Int. Cl. G01N 33/543; G01N 21/78; G01N 21/64; G01N 33/569; G01N 33/68; G01N 33/533; A61M 16/06; A41D 13/11; A41D 31/04

(52) U.S. Cl. CPC G01N 33/54306; G01N 21/78; G01N 21/6428; G01N 33/54387; G01N 33/56983; G01N 33/68; G01N 33/533; A61M 16/06; G01N 2333/165; A41D 13/11; A41D 31/04

(57) ABSTRACT

Provided are a diagnostic system and methods for detecting the presence or absence of one or more targets that represent virus infection, inflammatory diseases and/or respiratory disorders by optically and noninvasively observing changing in color of the diagnostic system upon binding of the antigen of interest. Exemplary diagnostic systems and methods include detection of SARS-CoV-2 virus S protein or receptor of advanced glycation end products (RAGE) for diagnosis of COVID-19 infection or inflammatory/respiratory diseases.

### FIELD OF THE INVENTION

The disclosure provides a diagnostic system and methods of manufacturing and using the system for detecting one or more targets of interest to make a diagnosis of viral infections, inflammatory diseases and/or respiratory disorders. In particular, the diagnostic system is capable of non-invasively detecting spike protein of SARS CoV-2 or inflammatory RAGE protein. The diagnostic system comprises a color changing moiety that allows optical observation of the presence or absence of a target of interest.

### BACKGROUND

Recent developments in virus outbreak as well as other contagious respiratory diseases have heightened awareness and needs for a rapid and efficient diagnostic system and more sophisticated biosensing methodologies. In addition to coronavirus, spreading of other infections, especially within community settings (e.g., hospitals, schools, nursing homes, etc.) require a quick, efficient detection of contaminants followed by prompt removal of the source of contamination. Some exemplary types of contagious infections include viral respiratory diseases (e.g., COVID-19), hospital-acquired infections (HAIs) that may further be sub-categorized into central line-associated bloodstream infections, catheter-associated urinary tract infections, ventilator-associated pneumonia and more. Often times, most contagious and deadly bacterial or viral infections are airborne. Being able to rapidly detect the presence of contamination is critical to controlling the spread of viruses and particularly important when confined in spaces (e.g., airplane, public transporation, etc.).

Conventional virus testing strategies include collecting specimen (e.g., a nasal swab test of COVID-19) for polymerase chain reaction (PCR) from symptomatic or asymptomatic persons. Some diseases may require blood tests and some may be detected by analysis of bodily fluids, including urine samples and other clinical samples. For other inflammatory or respiratory diseases, measuring biomarkers in lungs via invasive procedures such as bronchoscopy to obtain samples may be necessary, which may carry the associated costs, discomfort and risks. In order to reduce such limitations, non-invasively collecting potentially contaminated fluids or droplets out of the surface of an object or from the air for PCR analysis have been used. U.S. Pat. No. 9,617,582 to Milton describes a system and method for detecting a biomarker in exhaled breath condensate nanodroplets by noninvasively collecting exhaled breath condensate of a subject and analysing the nanodroplets. However, those system and method require additional analysing procedures, such as utilizing immunoquantitative polymerase chain reaction (PCR) to detect one or more target biomarkers. Even the most advanced PCR-based DNA amplification methods known in the art suffer from a number of drawbacks including high reagent costs, labor intensity and susceptibility to cross-contamination. Although those biomarker detection methods may have been readily incorporated into more accessible laboratory bench-top diagnostics, examples of non-invasive, relatively fast and user-friendly diagnostic systems that allow point-of-care are still not available. Conventional approaches encounter several additional limitations which include collecting contaminated samples for testing which can pose biocontamination or biohazard concerns, particularly for the clinicians or sample collectors who are in direct contact with the patients. Further, most of the currently developed testing methodologies take a relatively long time to generate diagnostic results.

Other non-PCR detection methods also exist and immunohistochemistry (IHC) is one of the most widely used clinical and diagnostic applications known in the art. However, the IHC-based detection methodologies also employ additional procedures and multiple probes and/or enzymes conjugations that are required for enzyme-promoted metallography or HRP-based chemiluminescence, which constrict the methods to the same limitations of being less capable of rapidly detecting or diagnosing the infections or diseases on the spot.

Faster analysis and complete automated non-invasive detection methodologies are highly desirable features that require continual improvement in any clinical analyzer. For example, a point-of-sample collection (POC) testing would enable the clinicians or users to diagnose and implement the detection in real-time by avoiding the need for large laboratory facilities for additional analysis steps.

As a result, conventional systems and methods for diagnosing and analysing virus infection and/or respiratory diseases fail to provide sufficient efficiency for detecting biomarkers in a non-invasive and one-step automated setting. Thus, there is a need in the art for an effective, noninvasive and user-friendly system and methodology which would open a new world of possibilities to the diagnosis and management of viral infection and any other respiratory and/or inflammatory disorders.

### SUMMARY OF THE INVENTION

The disclosure is directed to a diagnostic system and method for detection of pathogens or biomarkers as the diagnostic system is integrated into nonwoven or woven fibrous material which may be made into various forms of wearable or customizable systems. The disclosed systems and methodologies are suitable for the diagnosis of various infections, diseases and disorders, such as coronavirus infection, RAGE-related inflammatory and respiratory diseases and disorders, lung cancer, asthma, COPD, tuberculosis, influenza, HIV/AIDS related respiratory infections, and any other pathological conditions with identified biomarkers to be recognized by antibodies or molecules that are capable of binding to antigens or compounds of interest, such as oncological diseases, cardiovascular diseases, diabetes and in general severe disorders or deficits. One of the main advantageous features of the present invention is that the systems and methodologies are rapid and minimally or not at all invasive by providing chromatic output which may be easily observed by the naked eye or a minimal instrumentation infrastructure. The diagnostic system is also customizable and versatile in forms as it can be made into any style of wearable or covering fabric based on the intended use (e.g., face mask, bedding cover, surface cover, wipe, etc.).

One aspect of the invention is a diagnostic system which comprises at least one layer of fibrous material and a color changing moiety positioned on or within the at least one layer of fibrous material wherein the color changing moiety is comprised of gold particles (Au Ps), at least one linker linked to the Au Ps, an anchor protein linked to the at least one linker and at least one antibody or antibody fragment that is conjugated to the anchor protein, wherein the at least one antibody or antibody fragment has an affinity for an antigen of interest, and wherein the color changing moiety changes color when the at least one antibody or antibody fragment binds to the antigen of interest. In some embodiments, the at least one linker is covalently or non-covalently linked to the Au Ps and includes a molecule that has a sulfhydryl group and a carboxylic acid group. In some embodiments, the anchor protein is covalently or non-covalently linked to the last least one linker and at least one antibody or antibody fragment. In some embodiments, the system may include 11-Mercaptoundecanoic acid (MUDA) or cysteine (Cys) as a linker. In some embodiments, the anchor protein or molecule is selected from protein A, protein A ZZ domain, protein G, protein L, and fragments thereof. The at least one layer of fibrous material may be preferably nonwoven but can also be woven or any other fibrous material known in the art. In some embodiments, the antigen of interest may be any compound having a molecular weight of about 45 to 250 kDa. In preferred embodiments, the antigen of interest may be SARS-CoV-2 virus S protein or receptor of advanced glycation end products (RAGE).

In some embodiments, the Au Ps are immobilized on or grown on or bound to one or more fibers of the at least one layer of fibrous material. The diagnostic system may be configured as a face mask wearable by a human or non-human animal subject. In some embodiments, the diagnostic system may be configured as a garment, a bedding cover, a surface cover, a wipe that is for use on animal skin or on substrates other than animal skin. Another aspect of the invention is a method for detecting one or more antigens of interest. The method comprises the steps of bringing a fluid or surface which is contaminated with the one or more antigens into contact with the diagnostic system of the present disclosure; and detecting a change in color on at least one portion of the diagnostic system when the one or more antigens become bound to the at least one antibody or antibody fragment capable of recognizing the one or more antigens. In this method, the detection is preferably made by optical observation. In some embodiments, the detection may be made by an instrument or equipment that is configured for detecting or analyzing a color change. The optical observation and/or detection by an instrument may occur within about 10-30 minutes of binding of the one or more antigens to the at least one antibody or antibody fragment thereof. In some embodiments, the method may further include a step of assessing an amount of coupling between the antigen to antibody or antibody fragment capable of recognizing the one or more antigens. The method may also include a step of using the detecting instrument or equipment for the assessment. In such cases, the amount of coupling is calculated by comparing a degree of colorimetric change after contact with the fluid or surface with a known degree of colorimetric change after contact with a known amount of an antigen of interest.

In some embodiments, the antigen of interest is any protein that has a molecular weight of about 45 to 250 kDa. In preferred embodiments, the one or more antigens comprise SARS-CoV-2 virus S protein. In other preferred embodiments, the one or more antigens comprise receptor of advanced glycation end products (RAGE). In some embodiments, the fluid or surface brought into contact with the diagnostic system is or comprises breath exhaled from a subject, wherein the subject is human or non-human animal. The fluid or surface may also be or comprises a bodily discharge (e.g., sweat, urine, menstrual blood, feces, blood, tears, saliva and combinations thereof) of a subject. Preferably, the fluid of interest does not travel across or through a surface of the at least one layer of fibrous material.

In yet another embodiment, the invention is a method of manufacturing the diagnostic system and comprises the steps of synthesizing gold particles (Au Ps) on at least one piece of fabric by adding the at least one piece of fabric to a HAuCl₄ solution, optionally comprising a reducing agent, to nucleate Au⁰ directly on the fabric to form Au Ps fabric, conjugating at least one linker to the Au Ps nonwoven to form linker-coupled Au Ps nonwoven; linking at least one anchoring protein or molecule to the linker, and conjugating at least one antibody or antibody fragment capable of recognizing the one or more antigens to the at least one anchoring protein. In some embodiments, the at least one linker includes a molecule that has a sulfhydryl group and a carboxylic acid group. In some embodiments, the system may include 11-Mercaptoundecanoic acid (MUDA) or cysteine (Cys) as a linker. In preferred embodiments, the at least one piece of fabric is nonwoven. In other embodiments, the at least one piece of fabric is woven. The method of manufacturing the diagnostic system may further comprise: a step of pretreating the at least one piece of fabric in a solution containing a nucleating agent, wherein the nucleating agent may be ZnO nanoparticles (ZnO NPs) and/or a step of post-treating the at least one piece of Au Ps fabric in a HAuCl₄ solution, to further grow the nucleated Au Ps. In some embodiments, the at least one anchoring protein is selected from protein A, protein A ZZ domain, protein G, protein L, and fragments thereof.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

FIGS. 1A-B show (A) a magnified view of nonwoven fibrous material having (B) two types of fibers (e.g., rough fiber and smooth fiber) as an exemplary nonwoven fibrous material of diagnostic system in accordance with some embodiments of the present invention.

FIGS. 2A-C are illustrations showing an exemplary diagnostic system configured as a face mask having (A) a portion of the face mask (i.e., detection area) with a color changing moiety. The color changing moiety in the detection area changes color that indicates (B) a positive reaction or (C) a negative reaction in accordance with an embodiment of the present invention.

FIG. 3 is a profile view of the diagnostic system placed inside of a face mask.

FIG. 4 shows a profile view, a top view, and a cross-sectional view of an exemplary diagnostic system having honeycomb-patterned scaffolds in accordance with one embodiment of the present invention.

FIG. 5 is an illustration of a method of using an exemplary diagnostic system in accordance with an embodiment of the present invention FIG. 6 is an illustration and magnified view of the color changing moiety (i.e., bead panel) placed in either micro or macro texture fibrous material in accordance with various embodiments of the present invention.

FIGS. 7A-C are illustrations showing (A) a side view and a top view of an antibody, (B) gold nanoparticles, and (C) coronavirus as an exemplary virus with surface antigens.

FIG. 8 is an illustration showing a color changing moiety that comprises gold particles, a linker, and an antibody with antigen binding affinity in accordance with an embodiment of the present invention.

FIG. 9 is an illustration showing a color changing moiety that comprises gold particles, MUDA as an exemplary linker, protein A as an exemplary anchoring protein, and an antibody (IgG) with antigen binding affinity in accordance with an embodiment of the present invention.

FIGS. 10A-B are illustrations showing (A) multiple sets of color changing moieties of FIG. 9 and (B) release of the linker from the gold particles to expose the gold core-shell upon antigen-antibody binding, resulting in a color change in accordance with an embodiment of the present invention.

FIGS. 11A-B show various degrees of color change on nonwovens after nucleation of gold particles in different experimental conditions (i.e., the starting nonwoven is white).

FIGS. 12A-B show SEM images of gold particles as white dots on nonwovens after the nucleation of gold particles in different experimental conditions.

FIG. 13 shows a flow chart describing two principal steps of manufacturing the diagnostic system in accordance with an embodiment of the present invention.

FIGS. 14A-C show (A) assessment and optimization steps, (B) images obtained from the chrome test on a selected spot of the diagnostic system, comparing the untreated, treated with 5 mM gold nanoparticles, or treated with antibody and gold diagnostic systems in accordance with embodiments of the present invention, and (C) images obtained from the chrome test on another selected spot of the diagnostic system, comparing the untreated, treated with 5 mM gold nanoparticles, or treated with antibody and Ag diagnostic systems in accordance with embodiments of the present invention.

FIGS. 15A-B show (A) an illustration showing a color changing moiety that comprises Au particles (AuCl₃), a linker (Cys), and an anti-folate with folate binding affinity in accordance with an embodiment of the present invention and (B) images obtained from colorimetric chrome test after following the modified functionalization protocol in accordance with an embodiment of the present invention.

FIGS. 16A-B show laser confocal scanning microscopy (LSCM) images from the fluorescent test of (A) the samples with gold nanoparticles only in a brightfield view (left) and under a fluorescence view (right), and (B) the samples with gold nanoparticles conjugated with primary and secondary antibodies under fluorescence at 800 microns exposure (left) and 150 microns exposure (right) conditions.

FIG. 17 shows images of diagnostic systems of the present invention which are at various conjugation states (left to right): gold nanoparticles only, gold nanoparticles bound to MUDA, gold nanoparticles bound to MUDA, protein A and antibody, gold nanoparticles bound to MUDA, protein A, primary antibody and secondary antibody.

FIG. 18A-B show images of (A) the activated or unactivated pre-functionalized gold nanoparticles on nonwoven fibrous material that are optically observable and (B) validating fluorescent images of the activated or unactivated pre-functionalized gold nanoparticles in accordance with an embodiment of the present invention.

FIG. 19A-B show schemes of a color-changing moiety comprising Au particles, a linker (MUDA), protein A, and (A) the viral protein of SARS-CoV-2, the spike 1 protein (S1), and the antibody anti-protein S1 or (B) the antibody anti-virion SARS-CoV-2. The binding affinity is in accordance with an embodiment of the present invention and colorimetric change in the presence of the S1 or the virions within the breath is in accordance with an embodiment of the present invention.

### DETAILED DESCRIPTION

As used herein, the following terms have the meanings indicated.

The term “activate” or “positive reaction” refers to a condition affecting the antibody (or molecule capable of binding an antigen of interest) of the color changing moiety of the diagnostic system to bind to one or more antigens which results in a colorimetric change of the color changing moiety of the diagnostic system.

The term “aerosol” or “droplet” means a volume of liquid or solid particles that are small and lightweight sufficient to be suspended in air and float. The term “aerosol” may be used interchangeably with “droplet” in the present disclosure. The droplets may refer to large mucus or saliva particles heavier than air that fall toward the ground as soon as they are expelled. The droplets may take a wide variety of shapes; nonlimiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more surfaces where the droplet is placed.

The term “affinity” means the specific or non-specific intermolecular attraction of one molecule for another molecule or for a substrate, such as the attraction of an antibody for its corresponding antigenic molecules or pathogens.

The term “antibody” means a polypeptide that has affinity for an epitope of an antigen. An antibody can be a polyclonal antibody, a monoclonal antibody, an antibody fragment, and/or an engineered molecule capable of binding the corresponding member of a specific binding pair. Antibodies may be labeled or otherwise conjugated to molecules that facilitate direct or indirect detection of and/or quantification of the antibody. “Monoclonal antibody” as used in the present invention refers to the preparation of antibody molecules that have a common sequence of the heavy chain and light chain, in contrast to the preparations of “polyclonal” antibodies that contain a mixture of different antibodies. Monoclonal antibodies can be obtained in several new ways, such as phage, bacterial, yeast or ribosomal display, as well as classical methods, for example, antibodies obtained from hybridomas (e.g., antibodies secreted by a hybridoma obtained using a hybrid technique, such as the standard Kohler hybrid technique and Milstein ((1975) Nature 256: 495-497).

In a full-length antibody, each heavy chain consists of a variable region of the heavy chain (e.g., HCVR or VH) and a constant region of the heavy chain. The constant region of the heavy chain consists of three domains, CH1 CH2 and CH3. Each light chain consists of a variable region of the light chain (abbreviated herein as LCVR or VL) and a constant region of the light chain. The constant region of the light chain consists of one domain CL. The VH and VL regions can be further divided into hypervariability regions called complementarity determining regions (CDRs), between which there are regions that are more conservative and are called framework regions (FR). Each VH and VL consists of three CDRs and four FRs located from the amino end to the carboxyl end in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or a subclass. The term “anchor protein” or “anchor molecule” refers to a protein or a molecule comprising one or more natural IgG-binding domains, a hybrid or fusion protein comprising an IgG-binding domain, a mutant or variant of an IgG-binding domain, or a fragment of an IgG-binding domain. “Protein A” shall be taken to include a protein comprising one or more natural IgG-binding domains of protein A, a hybrid or fusion protein comprising an IgG-binding domain of a native or naturally-occurring protein A, or a mutant or variant of a native or naturally-occurring protein A that retains the ability of native protein A to bind. IgG, or a fragment of a native or naturally-occurring protein A that retains the ability of native protein A to bind IgG. “Protein G” shall be taken to include a protein comprising one or more natural IgG-binding domains of protein C, a hybrid or fusion protein comprising an IgG-binding domain of a native or naturally-occurring protein L, or a mutant or variant of a native or naturally-occurring protein G that retains the ability of native protein L to bind IgG, or a fragment of a native or naturally-occurring protein U that retains the ability of native protein G to bind IgG, “Protein L” shall be taken to include a protein comprising one or more natural IgG-binding domains of protein L, a hybrid or fusion protein comprising an IgG-binding domain of a native or naturally-occurring protein L, or a mutant or variant of a native or naturally-occurring protein L that retains the ability of native protein L to bind IgG, or a fragment of a native or naturally-occurring protein L that retains the ability of native protein L to bind. IgG.

The term “antigen”, “target molecule”, “biomarker”, “target polypeptide”, “target epitope” and the like may be interchangeably used in the present disclosure and refer to the molecule specifically bound by an antibody or antibody fragment or other molecule capable of binding target compound.

In some embodiments, a target molecule is a marker for a disease-causing agent (e.g., protein, DNA, RNA, peptide fragment, etc.), wherein the disease-causing agent is selected from the group of disease-causing organisms consisting of a virus, a bacterium, a mycoplasm, a fungus, a yeast, and other micro-organisms. In embodiments, a target molecule for a disease-causing agent is selected from the group consisting of Influenza A Matrix protein, Influenza H3N2, Influenza H1N1 seasonal, Influenza H1N1 novel, Influenza B, Streptococcus pyogenes (A), Mycobacterium Tuberculosis, Staphylococcus aureus (MR), Staphylococcus aureus (RS), Bordetella pertussis (whooping cough), Streptococcus agalactiae (B), Influenza H5N1, Influenza H7N9, Adenovirus B, Adenovirus C, Adenovirus E, Hepatitis b, Hepatitis c, Hepatitis delta, Treponema pallidum, HSV-1, HSV-2, HIV-1, HIV-2, Dengue 1, Dengue 2, Dengue 3, Dengue 4, Malaria, West Nile Virus, Trypanosoma cruzi (Chagas), Klebsiella pneumoniae (Enterobacteriaceae spp), Klebsiella pneumoniae carbapenemase (KPC), Epstein Barr Virus (mono), Rhinovirus, Parainfluenza virus (1), Parainfluenza virus (2), Parainfluenza virus (3), Parainfluenza virus (4a), Parainfluenza virus (4b), Respiratory syncytial virus (RSV) A, Respiratory syncytial virus (RSV) B, Coronavirus 229E, Coronavirus HKU1, Coronavirus OC43, Coronavirus NL63, Novel Coronavirus, Bocavirus, human metapneumovirus (HMPV), Streptococcus pneumoniae (penic R), Streptococcus pneumoniae (S), Mycoplasma pneumoniae, Chlamydia pneumoniae, Bordetella parpertussis, Haemophilus influenzae (ampic R), Haemophilus influenzae (ampic S), Moraxella catarrhalis, Pseudomonas spp (aeruginosa), Haemophilus parainfluenzae, Enterobacter cloacae (Enterobacteriaceae spp), Enterobacteraero genes (Enterobacteriaceae spp), Serratia marcescens (Enterobacteriaceae spp), Acinetobacter baumanii, Legionella spp, Escherichia coli, Candida, Chlamydia trachomatis, Human PapillomaVirus, Neisseria gonorrhoeae, plasmodium, and Trichomonas (vagin).

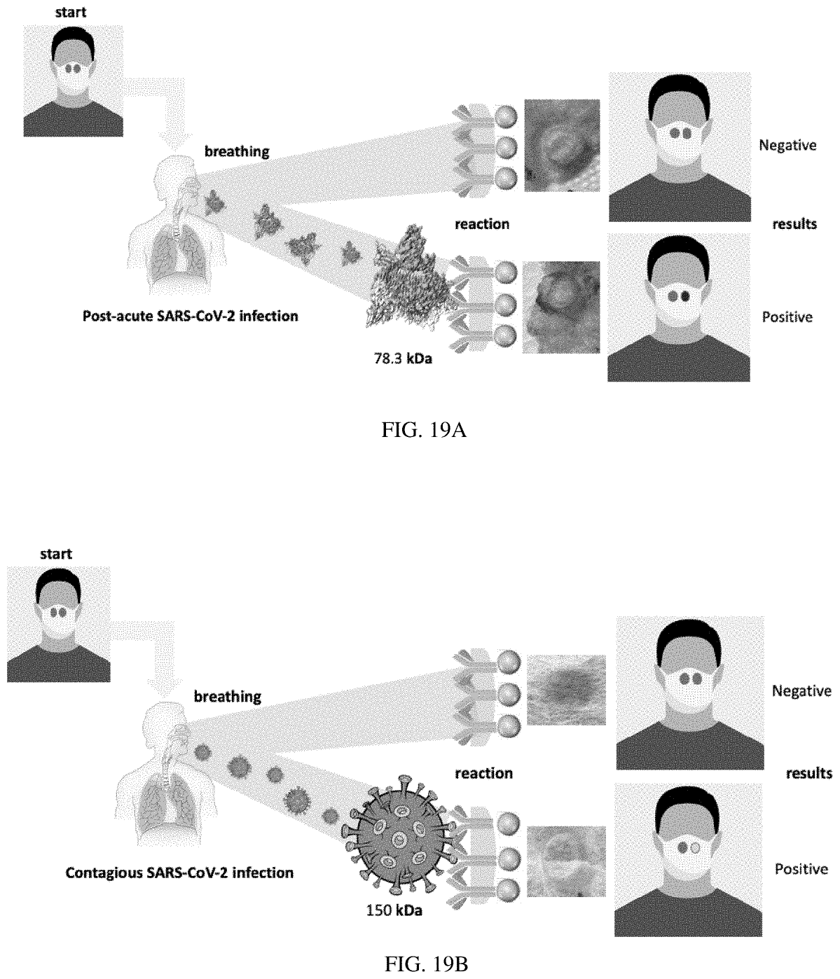

In some embodiments, an antigen or biomarker may be detected in an exhaled breath (e.g. FIGS. 19A-B), from a swab, in a blood sample or in other bodily fluid, wherein the antigen is a marker for inflammation and/or for a disease-causing agent. Such biomarker for inflammation is selected from receptor for advanced glycation end products (RAGE), prostaglandins, tumor necrosis factor alpha (TNF-α), interleukin-1 (IL-1), interleukin-8 (IL-8), interleukin-12 (IL-12), interferon gamma (IF-γ), bradykinin, complement system molecules, blood-clotting factors, C-reactive protein, erythrocyte sedimentation rate (ESR), white blood cell count, and morphological changes in blood and other cells, and such a disease marker.

The term “fibers” or “fibrous materials” described in the present invention may be any types of synthetic and/or natural fibers. In some embodiments, the fibers may be synthetic and/or cellulosic fibers. Exemplary fibers which can be used in the practice of the invention include but are not limited to: cotton, kapok, flax, ramie, kenaf, abaca, coir, hemp, jute, sisal, rayon, bamboo fiber, Tencel®, and Modal® fibers, glass fibers, basalt fibers, Kevlar® fibers, aramid fibers, polyester fibers (e.g., which can function both as a binder fiber but, depending on the polyester, as part of the nonwoven blend), wool (which may be obtained, for example, from one of the forty or more different breeds of sheep, and which currently exists in about two hundred types of varying grades), silk, rayon (a man-made fiber that may include viscose rayon and cuprammonium rayon), acetate (a man-made fiber), nylon (a man-made fiber), acrylic (a man-made fiber), triacetate (a man-made fiber), spandex (an elastomeric man-made fiber such as Lycra®), polyolefin/polypropylene (man-made olefin fibers), microfibers and microdeniers, lyocell (a man-made fiber), vegetable fiber (a textile fiber of vegetable origin, such as cotton, kapok, jute, ramie, polylactic acid (PLA) or flax), vinyl fiber (a manufactured fiber), alpaca, angora, carbon fiber (suitable for textile use); (t) glass fiber (suitable for textile use), raffia, ramie, vinyon fiber (a manufactured fiber), Vectran® fibers (manufactured fiber spun from Celanese Vectra® liquid crystal polymer), and waste fiber. Fibers are commercially available from sources known by those of skill in the art, for example, E. I. Du Pont de Nemours & Company, Inc. (Wilmington, Del.), American Viscose Company (Markus Hook, Pa.), Teijin Frontier Co., Ltd. (Osaka, Japan), Tintoria Piana USA (Cartersville, Ga.), and Celanese Corporation (Charlotte, N.C.).

In addition to the fibers described herein, other fibers (i.e., optional fibers) may be included in manufacturing the nonwovens or wovens to achieve properties or characteristics of interest (e.g., color, texture, etc.). The optional fibers may be present in sufficient amounts to provide a characteristic to the fibrous material such as softness, texture, appearance, resilience, and cost benefit. In some applications, the fibrous material may also include fabrics knitted or woven from different cellulosic fibers as described herein. In other embodiments, multiple layers of additional fibers may be added or attached to fibrous material. Other layers of fibers may include alginate, viscose, carboxymethyl chitosan, acylated chitosan, carboxymethyl cellulose, carboxylethyl cellulose, water insoluble cellulose alkyl sulfonate, bi-component, polyvinyl alcohol, polypropylene, polyethylene terephthalate, polyacrylonitrile, cross-linked acrylate copolymer, wood pulp and combinations thereof.

A “sample”, or “biological sample”, or “clinical fluid sample”, or “fluid” refers to a sample of fluid, tissue in fluid, secretion, or excretion obtained from a subject. A sample, biological sample, or clinical sample may be a sample of blood, serum, plasma, saliva, sputum, urine, gastric fluid, digestive fluid, tears, sweat, stool, semen, vaginal fluid, interstitial fluid, fluid derived from tumorous tissue, ocular fluids, mucus, earwax, oil, glandular secretions, spinal fluid, skin, cerebrospinal fluid from within the skull, tissue, fluid or material obtained or spilled from a nasal swab, a throat swab, a mouth swab (e.g., a cheek swab), a vaginal swab, or nasopharyngeal wash, biopsy fluid or material, placental fluid, amniotic fluid, cord blood, lymphatic fluids, cavity fluids, pus, microbiota obtained from a subject, meconium, breast milk, or other secretion or excretion. A sample may be a breath sample. The fluid sample may also be fluid obtained by washing a body cavity or surface of a subject or by washing a swab following the application of the swab to a body cavity or surface of a subject or a hair, a fingernail, ear wax, and other solid, semi-solid, or gaseous sample processed in a extraction buffer. The extraction buffer or an aliquot thereof may then be processed similarly to other fluid samples if desired. The sample may be obtained from a human or non-human animal. The sample may be obtained from a vertebrate, e.g., a bird, fish, or mammal, such as a rat, a mouse, a pig, an ape, another primate (including humans), a farm animal, a sport animal, or a pet. The sample may be obtained from a living or dead subject. The sample may be obtained fresh from a subject or may have undergone some form of pre-processing, storage, or transport.

The term “conjugate” refers to a compound having a molecule (for example, a biomolecule, such as an antibody) effectively coupled to another molecule (for example, a nanoparticle or a linker), either directly or indirectly, by any suitable means. In some examples, the molecule (such as an antibody) can be directly covalently coupled to a nanoparticle. In other examples, the molecule (such as an antibody) can be coupled to nanoparticles such as by using a “linker” molecule, so long as the linker does not significantly negatively affect the activity of the antibody or the function of the biomolecule. The linker preferably is bio-compatible. Common molecular linkers known in the art include a maleimide or succinimide group, streptavidin, neutravidin, biotin, or similar compounds. “conjugating”, “joining”, “bonding” or “linking” refers to coupling a first unit to a second unit. This includes, but is not limited to, covalently bonding one molecule to another molecule (for example, directly or via a linker molecule), noncovalently bonding one molecule to another (e.g., electrostatically bonding) (see, for example, U.S. Pat. No. 6,921,496, which discloses methods for electrostatic conjugation), non-covalently bonding one molecule to another molecule by hydrogen bonding, non-covalently bonding one molecule to another molecule by van der Waals forces, and any and all combinations of such couplings.

The term “linker” refers to a chemical moiety that connects one peptide to another, e.g., one antibody to another. Linkers can also be used to attach antibodies to labels or solid substrates. A linker can include amino acids. Linkers can be straight or branched, saturated or unsaturated carbon chains. They can also include one or more heteroatoms within the chain or at the termini of the chains. By “heteroatoms” is meant atoms other than carbon which are chosen from the group comprising of oxygen, nitrogen, sulfur, phosphorus, boron and halogen. In specific embodiments, linkers are carbon chains. The use of a linker may or may not be advantageous or needed, depending on the specific antibody pairs. Methods and techniques for the attachment of a linker to an antibody are known in the art. For a treatise on this subject, the reader is referred to Bioconjugate Techniques, G. Hermanson, Academic Press, 1996.

The term “colorimetric detection” refers to techniques of quantifying or otherwise observing colored diagnostic system. The term also refers to any method of visually detecting such colored diagnostic system and/or the change in color of the diagnostic system of the present disclosure. Methods may include visual observation, absorbance measurements, or fluorescence measurements, among others. Generally, colorimetric detection is meant to detect colors within the wavelength range of between 380-700 nm.

The term “contacting” refers to a placement that allows association between two or more moieties, particularly direct physical association, for example in solid form and/or in liquid form and/or in gaseous form (for example, the placement of the diagnostic system to a fluid sample on a surface, such as a biological fluid sample affixed to a surface, such as placing antigen contaminated exhaled breath in contact with an antibody or a probe containing diagnostic system).

The term “severe acute respiratory syndrome coronavirus 2 (SARS-CoV-2)” refers to virus comprising a virion with 50-200 nanometers in diameter and a genomic size of about 30 kilobases, encoding multiple structural proteins, such as the S (spike), E (envelope), M (membrane) and N (nucleocapsid), and non-structural proteins. Coronaviruses are a group of related RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause SARS, MERS, and COVID-19. Coronaviruses constitute the subfamily Orthocoronavirinae, in the family Coronaviridae, order Nidovirales, and realm Riboviria. Coronaviruses have four genera: alpha-, beta-, gamma-, and delta-coronaviruses. They are enveloped viruses with a positive-sense single-stranded RNA genome and a nucleocapsid of helical symmetry. The genome size of coronaviruses ranges from approximately 26 to 32 kilobases. Exemplary coronaviruses that may be detected with the systems and methods of the disclosure include, but are not limited to, SARS-Cov, SARS-Cov-2, MERS-Cov, HCoV-0C43, HCoV-HKU1, HCoV-229E, and HCoV-NL63. Coronaviruses (CoVs) are positive single-stranded (+ss) RNA viruses that are classified within the family Coronaviridae. The infectious bronchitis virus (IBV) was the first-discovered CoV that caused an outbreak of respiratory illness in chickens in the 1930s. Reports on mouse hepatitis virus (MHV) and transmissible gastroenteritis virus (TGEV) that infected mice and pigs, respectively, followed in the 1940s. In humans, HCoV-229E and HCoV-0C43, were identified in the 1960s as the causative agents of mild respiratory diseases that present as common cold. Since then, five other HCoVs were identified at different times, including Severe Acute Respiratory Syndrome-CoV (SARS-CoV) in 2003, HCoV-NL63 in 2004, HCoV-HKU1 in 2005, Middle East Respiratory Syndrome-CoV (MERS-CoV) in 2012, and the most recently discovered Severe Acute Respiratory Syndrome-CoV-2 (SARS-CoV-2) in December 2019.

Since SARS-CoV-2 shares 80% sequence homology with SARS-CoV-1, the antibodies of the diagnostic system, in accordance of an exemplary embodiment of the present invention, against S protein in SARS-CoV-2 may also be compatible with SARS-Cov-1. Thus, other virus types such as SARS-CoV (i.e., SARS-CoV-1) and MERS-CoV that are similar in virion structure may also be subjected to the present invention. As used herein, the term “S protein” or “Spike protein” is used to refer to a knoblike structured (i.e., spikes) peplomer, which is composed of glycoprotein to project from the lipid bilayer of the surface envelope of an enveloped virus. The “spike protein” or “S protein” is interchangeably referred to a protein and/or a glycoprotein. Furthermore, the sequences encoding the SARS-CoV-2 glycoprotein may also be referred to as a peptide or amino acid sequence.

The term “detect” refers to an action determining if an agent (such as a signal or particular target molecule) is present or absent, for example, in a sample. In some examples, this can further include quantification. “Detecting” refers to any method of determining if something exists, or does not exist, such as determining if a target molecule is present in a biological sample. For example, “detecting” can include using a visual or a mechanical device to determine if a sample displays a specific characteristic. In certain examples, detection refers to visually observing an antibody bound to a target molecule, or observing that an antibody does not bind to a target molecule.

The term “direct linkage” refers to a coupling or conjugation of two molecules without an intervening linker. In some examples, a direct linkage is formed when an atom of a first molecule (such as an antibody) bonds to an atom of a second molecule (such as a nanoparticle). In some examples, the direct linkage is a covalent bond, such as a metal-thiol bond (for example, a gold-thiol bond).

The term “nanoparticle” refers to a nanoscale particle with a size that is measured in nanometers, for example, a nanoscopic particle that has at least one dimension of less than about 200 nm. Examples of nanoparticles include, by way of example and without limitation, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In particular examples, a nanoparticle is a metal nanoparticle (for example, a nanoparticle of gold, palladium, platinum, silver, copper, nickel, cobalt, iridium, or an alloy of two or more thereof). Nanoparticles can include a core or a core and a shell, as in core-shell nanoparticles. The term “nonwoven” described herein, is a manufactured sheet, web, or batt of natural and/or man-made fibers or filaments that are bonded to each other by any of several means. Manufacturing of nonwoven product is well described in “Nonwoven Textile Fabrics” in Kirk-Othmer Encyclopedia of Chemical Technology, 3^{rd} Ed., Vol. 16, July 1984, John Wiley & Sons, p.72-124 and in “Nonwoven Textiles”, November 1988, Carolina Academic Press. Web bonding methods include mechanical bonding (e.g., needle punching, stitch, and hydro-entanglement), chemical bonding using binder chemicals (e.g., saturation, spraying, screen printing, and foam), and thermal bonding using binder fibers with low-melting points. Two common thermal bonding methods are air heating and calendaring. In some embodiments, hot-air thermal bonding using low-melt binder fibers may be employed to manufacture the nonwoven. In these embodiments, the low-melt binder fibers melt at a lower temperature than the melting point or decomposition temperature of other synthetic or cellulosic fibers (e.g., one or more antimicrobial treated fibers and/or untreated cellulosic fibers) so that the binder fibers serve to hold the one or more antimicrobial treated synthetic or cellulosic fibers and/or the untreated synthetic or cellulosic fibers together in a nonwoven. Preferred melting temperatures of binder fibers and cellulosic fibers are described in U.S. Pat. No. 10,508,370, herein incorporated by reference.

The term “polypeptide” or “protein” refers to a polymer in which the monomers are amino acid residues which are joined together through amide bonds. When the amino acids are alpha-amino acids, either the L-optical isomer or the D-optical isomer can be used. The terms “polypeptide,” “peptide,” or “protein” as used herein are intended to encompass any amino acid sequence and include modified sequences such as glycoproteins. The term “polypeptide” or “protein” is specifically intended to cover naturally occurring proteins, as well as those which are recombinantly or synthetically produced.

The term “reducing agent” refers to an element or compound that reduces another species. In reducing another species, the reducing agent becomes oxidized, and is an electron donor. In particular examples, reducing agents include, but are not limited to dithiothreitol (DTT), isopropanol, glycine, 4-(2-hydroxyethyl)-1-piperazineethanesulfonic acid (HEPES), and sodium thiosulfate.

The term “receptor for advanced glycation end products (RAGE)” refers to human RAGE, also called “hRAGE” or “huRAGE”. Unless otherwise indicated, the term “RAGE” also encompasses RAGE molecules isolated or derived from other non-human species, for example, rodents, like mice or rats; or RAGE molecules of a bull. The term “sRAGE” refers to a soluble form of RAGE derived from the extracellular domain of RAGE. RAGE is also found in different species, and thus includes species variants. RAGE is highly expressed in the embryonic central nervous system. In adult tissues, RAGE is expressed at low levels in multiple tissues including endothelial and smooth muscle cells, mononuclear phagocytes, pericytes, microglia, neurons, cardiac myocytes and hepatocytes. The expression of RAGE is upregulated upon ligand interaction. Depending on the cellular context and interacting ligand, RAGE activation can trigger differential signaling pathways that affect divergent pathways of gene expression. RAGE activation modulates varied essential cellular responses (including inflammation, immunity, proliferation, cellular adhesion and migration) that contribute to cellular dysfunction associated with chronic diseases such as diabetes, cancer, amyloidoses and immune or inflammatory disorders and other proliferative and degenerative diseases, including neurodegenerative diseases and endometriosis. RAGE receptors are implicated in induction of cellular oxidant stress responses, including via the RAS-MAP kinase pathway and NF-_{κ}B activation.

The term “specifically binds” refers to the binding of an agent that preferentially binds or substantially only binds to a defined target (such as an antibody to a specific antigen or a nucleic acid probe to a specific nucleic acid sequence). With respect to an antigen, “specifically binds” refers to the preferential association of an antibody or other ligand, in whole or part, with a specific polypeptide or molecule.

The term “point of service” (abbreviated POS) and “point of service system,” as used herein, refer to a location, and a system at that location, that is capable of providing a service (e.g. testing, monitoring, treatment, diagnosis, guidance, sample collection, verification of identity (ID verification), and other services) at or near the site or location of the subject. A service may be a medical service, and may be a non-medical service. In some situations, a POS system provides a service at a predetermined location, such as a subject's home, school, or work, or at a grocery store, a drug store, a community center, a clinic, a doctor's office, a hospital, etc. A POS system can include one or more point of service devices. In some embodiments, a POS system is a point of care system. In some embodiments, the diagnostic system of the present disclosure provides POS that includes steps of sample collection, testing, monitoring, diagnosing and identifying a subject's medical condition.

The present invention provides a diagnostic system having a color changing moiety that comprises antibody-metallic particles conjugates capable of changing color upon antigen-antibody binding. The present invention further provides a method of using the diagnostic system for detecting a target molecule (e.g., a protein or nucleic acid molecule of interest). A method of manufacturing the diagnostic system is also disclosed herein.

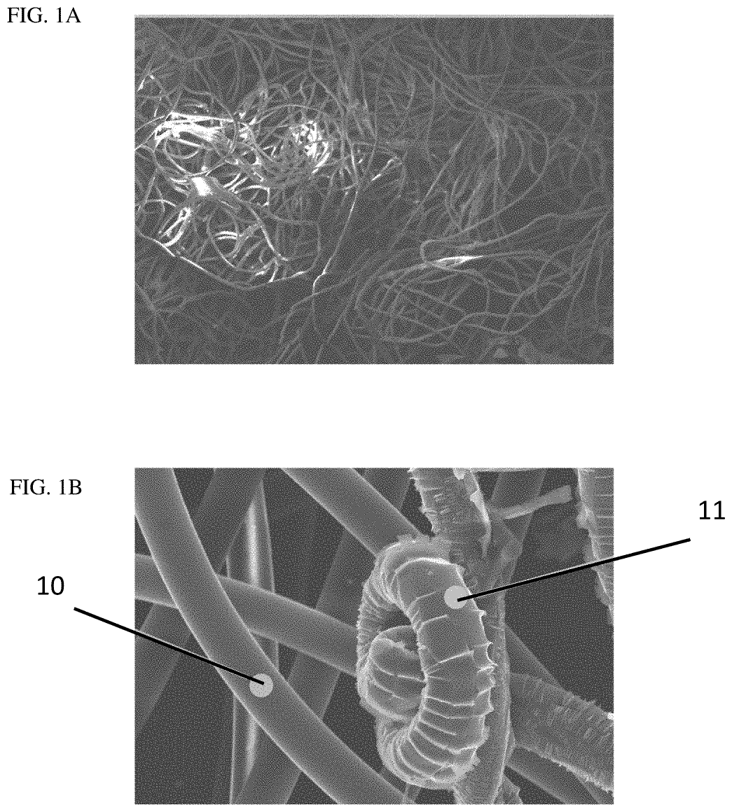

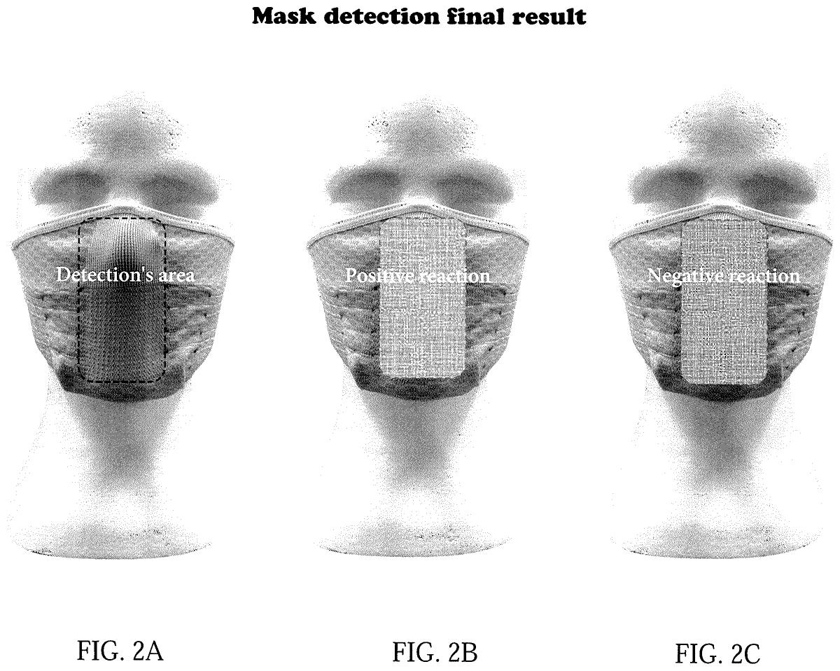

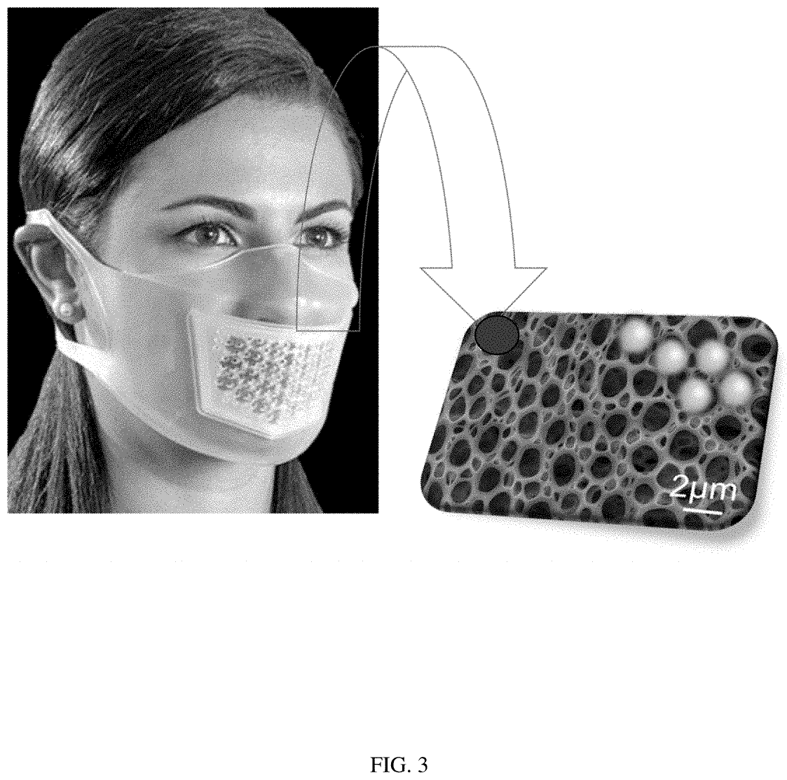

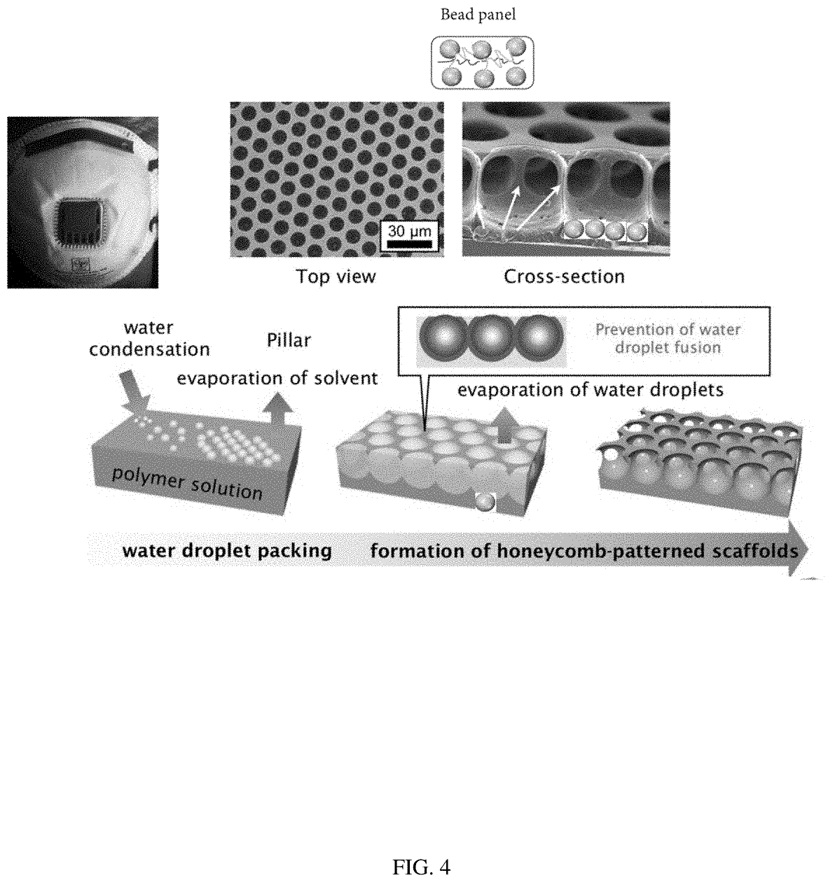

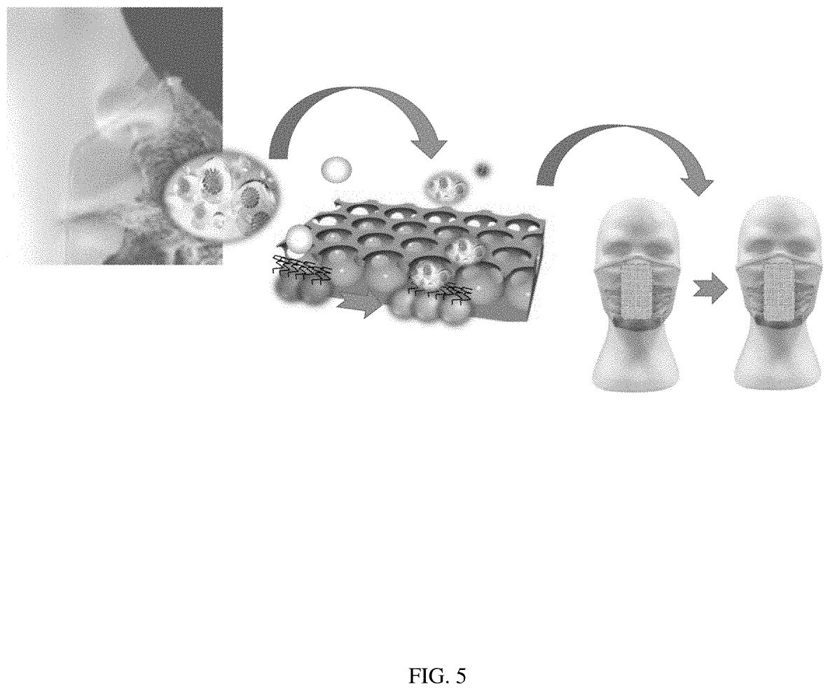

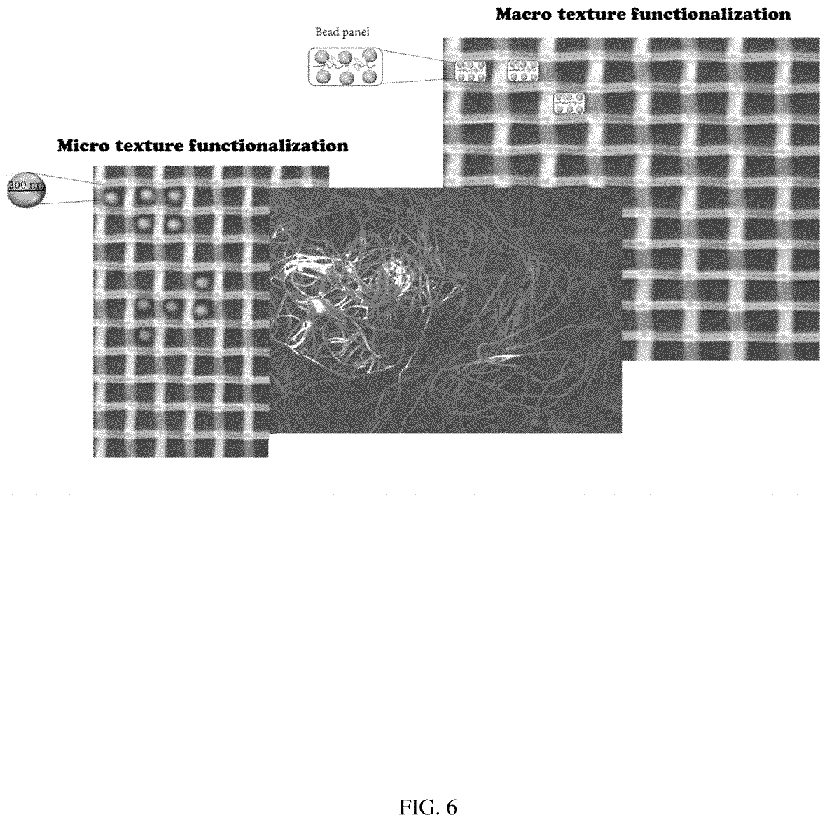

The diagnostic system comprises at least one layer of fibrous material and a color changing moiety positioned on or within the at least one layer of fibrous material. The fibrous material may be manufactured into a nonwoven or woven fabric. In preferred embodiments, the fibrous material is nonwoven comprising both smooth 10 and rough fibers 11, as shown in FIGS. 1A-B. In particular example where the diagnostic system is configured as a face mask, the diagnostic system allows optical observation of the presence or absence of an antigen of interest in an exhaled breath sample from a subject (FIGS. 2A-C and 19A-B). The face mask is designed to be worn on a wearer's face, covering either some portions or the entirety of the face, and includes at least a mask body and a pair of ear straps. The mask body covers at least the mouth and nose of a wearer, as shown in FIG. 3. Further, multiple layers of nonwovens may be used to fabricate the face mask diagnostic system. For example, a first fiber sheet and a second fiber sheet having the same or different color changing moiety may be bonded, attached, woven, laid on top of each other to form a face mask. Another example, as shown in FIG. 3, the diagnostic system of the present disclosure may be inserted or attached to another system. In other embodiments, the diagnostic system of the present disclosure may further comprise a section that enclose the color changing moiety in a polymerized bedding (FIG. 4). The polymerized bedding may be manufactured by packing water droplets followed by evaporation of the water droplets to form honeycomb-patterned scaffolds where the color changing moiety are placed and/or immobilized. In order to prevent further contamination, the diagnostic system in the form of a face mask may be configured to be impermeable so that the antigen of interest does not travel across or through a surface of the at least one layer of the fibrous material and/or the polymerized bedding inserted into the diagnostic system of the present disclosure (FIG. 5). Depending on the texture of the fibrous material, the color changing moiety of the diagnostic system may be individually placed and functionalized in the fibrous material of micro texture or functionalized in groups of multiple color changing moiety when placed in the fibrous material having a macro texture (FIG. 6).

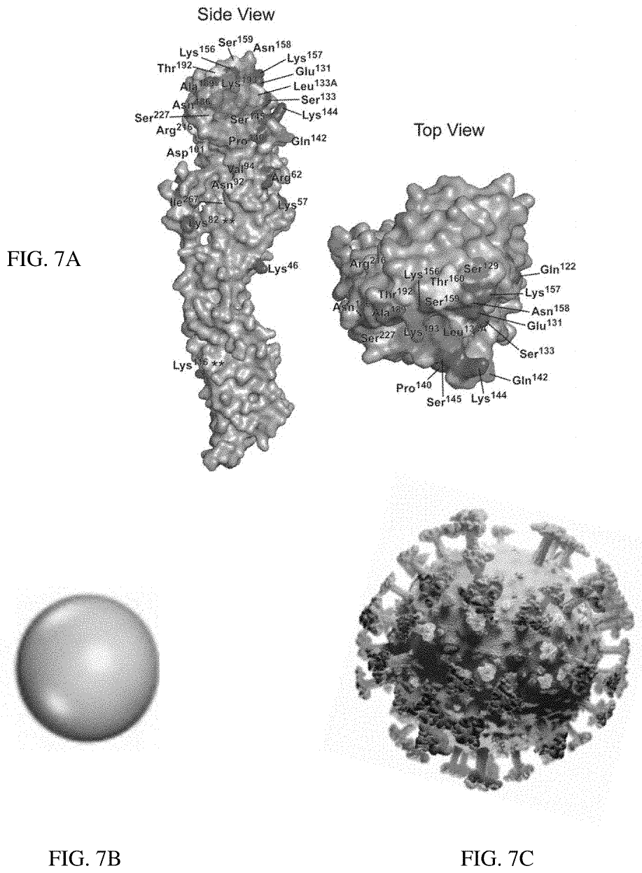

The color changing moiety described herein include antibody or any molecule capable of detecting a target compound (FIGS. 7A-C) and metallic particles that are either indirectly or directly linked to each other. In some embodiments, the particles (FIG. 7B) are metallic nanoparticles. Examples of metallic nanoparticles include nanoparticles made of gold, palladium, platinum, silver, copper, nickel, cobalt, or iridium, ruthenium, rhodium, osmium or iron. In some embodiments, the particles are an alloy of two or more metals. In other examples, the particles is a core-shell nanoparticles, having a metal core with a shell of a different metal (e.g., a silver nanoparticle including a gold shell). In preferred embodiments, the particles are gold particles (Au Ps) or gold nanoparticles (Au NPs). The gold nanoparticles may include a metal core having about 10-200 gold atoms, for example, about 100-200 gold atoms, or about 100-150 gold atoms. In some embodiments, the particles are gold particles (Au Ps) having 200 gold atoms or more, 300 gold atoms or more, 500 gold atoms or more, 1000 gold atoms or more, 2000 gold atoms or more at its metal core. The particles of this present disclosure may have a diameter of from about 0.5 nm or more, 1 nm or more, 10 nm or more, 100 nm or more, 1 um or more, 5 μm or more, 10 um or more, 50 um or more, 100 μm or more, 200 μm or more, with or without a crosslinked antibody. In preferred embodiments, the metallic particles or nanoparticles are immobilized on or grown on or bound to one or more fibers of the at least one layer of fibrous material.

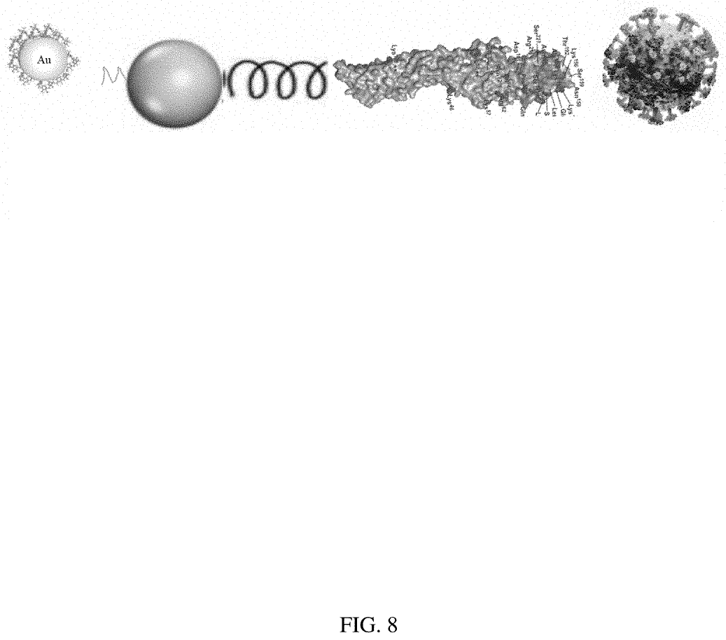

Referring to FIG. 8, an exemplary antibody-metallic particles conjugate of the color changing moiety is shown. In some embodiments, the color changing moiety comprises gold nanoparticles, a linker, protein A, and an antibody, which are crosslinked in such order. As shown in FIG. 8, in some embodiments, the gold nanoparticles are linked to protein A and the linkage between the gold nanoparticles and protein A is achieved by a chemical linker. In some embodiments, the gold particles or nanoparticles may be linked to any anchoring protein or molecule that comprises IgG-binding domains. For example, the anchoring protein may be selected from protein A, protein A ZZ domain, protein G, protein L, and fragments thereof.

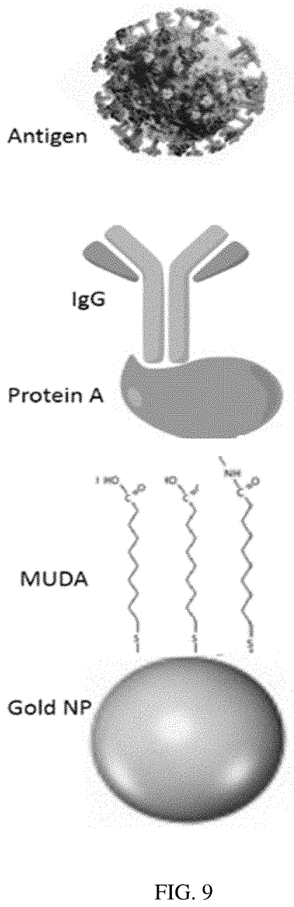

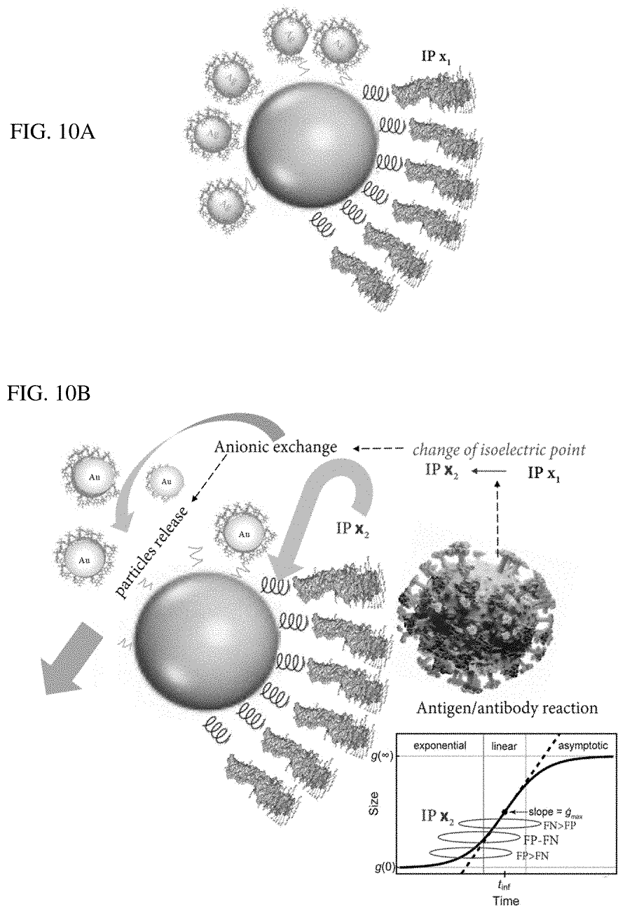

In some embodiments, the gold nanoparticles and protein A are linked by a linker with a carbon chain length in the range of 5 to 50 carbons, preferably 6 to 30 carbons, more preferably 7 to 20 carbons. In preferred embodiments, the linker has a sulfhydryl moiety and carboxylic acid moiety that readily bind to the gold surface and the amino groups of the anchoring protein, respectfully. In specific embodiments, as shown in FIG. 9, the linker is 11-mercaptoundecanoic acid (MUDA). In some embodiments, the linker is cysteine (Cys). For example, MUDA may link the gold particles to protein A to assure the reciprocal covalent interaction, thereby preventing other covalent interaction of the protein A to other available surfaces on the fibers or fibrous materials. The presence of the MUDA introduces a distance between gold nanoparticles and protein A.

In some embodiments, the anchoring protein (e.g., protein A) is used to position or fix the conjugated antibody or any molecule that is capable of recognizing the one or more targets (i.e., immunoglobulin, Ig) into an appropriate orientation that exposes the active site of the antibody free to react with an antigen of interest. The disclosed color changing moiety may include at least one gold particles or nanoparticles linked to an antibody or antibody fragment. In these embodiments, the antibody can include monoclonal or polyclonal antibodies, such as IgA, IgD, IgE, IgG, or IgM. In some embodiments, antibody fragments include, without limitation, proteolytic antibody fragments (such as F(ab′)₂ fragments, Fab′ fragments, Fab′-SH fragments, and Fab fragments as are known in the art), recombinant antibody fragments (such as sFv fragments, dsFv fragments, bispecific sFv fragments, bispecific dsFv fragments, F(ab)′2 fragments, single chain Fv proteins (“scFv”), and disulfide stabilized Fv proteins (“dsFv”)). In other examples, the antibody can include diabodies, triabodies, and camelid antibodies; genetically engineered antibodies (such as chimeric antibodies, for example, humanized murine antibodies); heteroconjugate antibodies (such as, bispecific antibodies); and combinations thereof. In particular examples, the antibody includes so-called “secondary antibodies,” which include polyclonal antibodies with specificity for immunoglobulin (for example, IgG, IgA, or IgM) from a particular species (such as rabbit, goat, mouse, chicken, sheep, rat, cow, horse, donkey, hamster, guinea pig, or swine). In some examples, the antibody is a rabbit anti-goat IgG, a goat anti-rabbit IgG, whole human IgG, or mouse or rat antibodies. In one example disclosed herein, the antibody is a rabbit anti-goat IgG. In other examples, the antibody includes an anti-hapten antibody (such as an anti-dinitrophenyl (DNP) antibody, an anti-digoxigenin (DIG) antibody, an anti-fluorescein antibody, an anti-biotin antibody, or an anti-benzofurazan antibody).

In some embodiments, the antigen of interest is spike protein of SARS CoV-2. In some embodiments, the antigen of interest is inflammation RAGE protein. In other embodiments, the antigen of interest may be any other biomarker of any infection or condition. For example, the antigen of interest or biomarkers may be any proteins or molecules (e.g., chemokines, interleukins, cytokines, etc.) with a molecular weight over 40 kDa, preferably over 45 kDa, more preferably 50 kDa. In preferred embodiments, the antigen of interest is a protein having a molecular weight of about 40 to 350 kDa, preferably 45 kDa to 300 kDa, more preferably 50 to 250 kDa. In other embodiments, when the molecular weight of the antigen of interest is below 50 kDa, other validating assays may be concurrently or sequentially used. Some exemplary antigens (e.g., infectious disease biomarkers) are listed in U.S. Pat. No. 9,916,428, herein incorporated by reference. More exemplary antigenic cytokines or other small molecules are listed in U.S. Pat. Application 2018/0195960.

The diagnostic system of the present disclosure may be configured as a face mask, a garment, a surface cover, a wipe, a bedding cover, and the like. In some embodiments, the diagnostic system is configured as a wipe. The wipe may be configured for use on animal skin or on substrates other than animal skin. In some embodiments, the diagnostic system is configured as a surface cover. In some embodiments, the diagnostic system is configured as a garment. In some embodiments, the diagnostic system is configured as a bedding cover. The fibrous materials comprising the color changing moiety may be manufactured into bedding surface coverings and/or bedding materials may be used as mattress encasings, mattress covers, mattress toppers, mattresses, pillows, pillowcases, duvet cases, bedsheets, etc. As used herein, the terms “bedding surface coverings”, “beddings”, “surface coverings”, “bedding applications” and “bedding materials” may be used interchangeably. Such beddings may further include one of the following materials: a knit polyester, a knit polyester with a polyurethane backing, a spunbond/meltblown/spunbond olefin, a woven polyester, a woven cotton-polyester blend, and combinations thereof. Similarly, as it is apparent to one with skill in the art, the diagnostic system may be used to fabricate any types of surface covers (e.g., seat covers in public transportation, bench covers, desk covers, medical device covers, doorknob covers, etc.). In addition, the antimicrobial nonwovens, alone or in combinations with multiple layers of nonwovens or other woven materials, may be manufactured into diagnostic pillows and/or cushions, as well as pillow/cushion coverings. In some embodiments, the diagnostic system is used to fabricate additional products such as, but not limited to, facial tissues (e.g., Kleenex® tissues), diapers, feminine products (e.g., panty liners, pads, tampons, etc.), furniture, animal beds, pet beds, bandages (e.g. Band-Aid® bandages), etc.

In some embodiments, one or more other natural and safe antimicrobial chemicals (e.g., silver nitrate, copper, copper nitrate, zinc, nano-silver, chitosan, triclosan, quaternary ammonium compounds, polybiguanides, etc.) may be used in the fibrous material of the diagnostic system.

In preferred embodiments, the diagnostic system of the present disclosure provides a single reaction (i.e., antibody-antigen recognition) based response. The diagnostic system provides an on-spot detection, in other words, all of the required diagnostic steps, such as collection, interaction, reaction, response and detection, take place within or on the spot where the antigen of interest contacts the color changing moiety of the diagnostic system. Thus, the diagnostic system may not include a separate fluid handing system that aids to transport, dilute, extract, aliquot, mix fluids of interest. However, in other embodiments, the diagnostic system may comprise a fluid handling or transferring system that may comprise an embedded or detachable units of fluid handling system that may aid in performing, transport, dilution, extraction, aliquoting, mixing and other actions with a fluid sample. Although not required, the diagnostic system may include additional microfluidic systems that allow droplets or liquid or fluids of sample to travel or flow in either continuous-flow or discrete-flow channel in the system. The system may also comprise a flow system within channels or in a channel-less architecture. In preferred embodiments, the diagnostic system allows point-of-sample collection and detection without additional biochemical analysis and/or microfluidic systems.

Another aspect of the disclosure provides a method for detecting one or more antigens. The method comprises the steps of bringing a fluid or surface which is contaminated with the one or more antigens into contact with the diagnostic system described above; and detecting a change in color on at least one portion of the diagnostic system when the one or more antigens become bound to the at least one antibody. As shown in FIGS. 5A-B, when the one or more antigens are bound to the at least one antibody, the resulted changes in isoelectric point promotes anionic exchange to expose the originally covered, clustered, or aggregated metallic particles or nanoparticles, thus allowing a change in color on at least one portion of the diagnostic system where the antigen and antibody interaction occurs. The fluid of interest in such diagnostic system does not travel across or through a surface of the at least one layer of fibrous material, thus preventing cross-contamination as well as providing a POC diagnostic method of use. In some embodiments, the antigen of interest is spike protein of SARS CoV-2. In some embodiments, the antigen of interest is inflammation RAGE protein. In other embodiments, the antigen of interest may be any other biomarker any infection or condition. In particular examples, the antigen of interest or biomarkers may be any proteins or molecules (e.g., chemokines, interleukins, cytokines, etc.) with a molecular weight over 40 kDa, preferably over 45 kDa, more preferably 50 kDa. In preferred embodiments, the antigen of interest is a protein having a molecular weight of about 40 to 350 kDa, preferably 45 kDa to 300 kDa, more preferably 50 to 250 kDa. The antigen of interest or biomarkers may be present in the fluid or surface brought into contact with the diagnostic system. In some embodiments, the fluid or surface brought into contact with the diagnostic system is or comprises breath exhaled from a subject. In some embodiments, the fluid or surface is or comprises a bodily discharge (e.g., sweat, urine, menstrual blood, feces, blood, tears, saliva and combinations thereof) of a subject. A subject of the present invention is preferably a mammal. Such mammal can be, for example, a human, or a non-human animal such as a primate (e.g., a monkey, chimpanzee, etc.), a domesticated animal (e.g., dog, cat, horse, etc.), farm animal (e.g., goat, sheep, pig, cattle, etc.), or laboratory animal (e.g., mouse, rat, etc.). Preferably, a subject is a human.

The detecting method may be performed in a short period of time. The diagnostic system may be capable of performing all steps of a method on a single sample in a short amount of time. For example, in an exemplary embodiment of face mask diagnostic system, from breath sample exposure from a subject to detecting a disease marker, or to detecting multiple disease markers, or to detecting changes in color of the color changing moiety of the diagnostic system may take about 3 hours or less, 2 hours or less, 1 hour or less, 50 minutes or less, 45 minutes or less, 40 minutes or less, 30 minutes or less, 20 minutes or less, 15 minutes or less, 10 minutes or less, 5 minutes or less, 4 minutes or less, 3 minutes or less, 2 minutes or less, or 1 minute or less. For example, from breathing into the diagnostic system from a subject to transmitting data regarding, and/or to analysis of, a sample or samples may take about 3 hours or less, 2 hours or less, 1 hour or less, 50 minutes or less, 45 minutes or less, 40 minutes or less, 30 minutes or less, 20 minutes or less, 15 minutes or less, 10 minutes or less, 5 minutes or less, 4 minutes or less, 3 minutes or less, 2 minutes or less, or 1 minute or less. In preferred embodiments, the optical observation and/or detection by an instrument may occur within about 2-90 minutes, preferably 5-60 minutes, more preferably 10-30 minutes of binding of the one or more antigens to the at least one antibody. Thus, as used herein, a “short period of time” refers to a period of time of about 5 hours or less, or about 4 hours or less, or about 3 hours or less, or about 2 hours or less, or about 1 hour or less, or about 50 minutes or less, or about 40 minutes or less, or about 30 minutes or less, or about 20 minutes or less, or about 10 minutes or less, or about 5 minutes or less. A short period of time may be determined with respect to an initial time; the initial time may be the time at which the antibody of the diagnostic system recognizes at least one antigen of interest; or the initial time may be the time at which a fluid sample is in contact with the diagnostic system for the analysis of the sample.

Additional chemical or biochemical reaction or other processing step may be performed in order to confirm and validate the optical observation. For example, the method may further comprise a step of assessing an amount of antigen-antibody coupling. The amount of antigen-antibody coupling is calculated by comparing a degree of colorimetric change after contact with the fluid or surface with a known degree of colorimetric change after contact with a known amount of an antigen of interest. Examples of steps, tests, or assays that may be prepared or run by the device may include, but are not limited to immunoassay, nucleic acid assay, receptor-based assay, cytometric assay, colorimetric assay, enzymatic assay, electrophoretic assay, electrochemical assay, spectroscopic assay, chromatographic assay, microscopic assay, topographic assay, calorimetric assay, turbidimetric assay, agglutination assay, radioisotope assay, viscometric assay, coagulation assay, clotting time assay, protein synthesis assay, histological assay, culture assay, osmolarity assay, and/or other types of assays, centrifugation, separation, filtration, dilution, enriching, purification, precipitation, pulverization, incubation, pipetting, transport, cell lysis, or other sample preparation action or steps, or combinations thereof. Steps, tests, or assays that may be prepared or run by the device may include imaging, including microscopy, cytometry, and other techniques preparing or utilizing images. Steps, tests, or assays that may be prepared or run by the device may further include an assessment of histology, morphology, kinematics, dynamics, and/or state of a sample, which may include such assessment for the antibody and antigen interaction.

The diagnostic system may further be electronically coupled to a separate computer system including a processor, input and output devices, data storage medium and other components. This arrangement is particularly useful in the assessment of the amount of antigen-antibody coupling, in which the computer system is programmed to operate calculating the amount of antigen-antibody coupling. The computer system may calculate the amount by comparing with a degree of colorimetric change after contact with the fluid or surface with a known degree of colorimetric change after contact with a known amount of an antigen of interest. The processor of the computer system may further be connected to a control user interface (e.g., cellular phone, smartphones, location-aware portable phones, tablet, laptop, etc.) and transmit instructions to the computer system to calculate or display the colorimetric diagnosis, to read or store the data in memory or sensors, and the like.

Another aspect of the disclosure provides a method of manufacturing the diagnostic system described above and comprises the steps of synthesizing gold particles (Au Ps) on at least one piece of fabric by adding the at least one piece of fabric to a HAuCl₄ solution, optionally comprising a reducing agent, to nucleate Au⁰ directly on the fabric to form Au Ps fabric, conjugating at least one linker to the Au Ps nonwoven to form linker-coupled Au Ps nonwoven; linking at least one anchoring protein or molecule to the linker, and conjugating at least one antibody or antibody fragment capable of recognizing the one or more antigens to the at least one anchoring protein or molecule. In some embodiments, the at least one linker includes a molecule that has a sulfhydryl group and a carboxylic acid group. In some embodiments, the system may include 11-Mercaptoundecanoic acid (MUDA) or cysteine (Cys) as a linker. The at least one anchoring protein is selected from protein A, protein A ZZ domain, protein G, protein L, and fragments thereof. The reducing agent may be isopropanol, glycine, methanol, 4-(2-hydroxyethyl)-1-piperazineethanesulfonic acid (HEPES), but also other reducing compounds, side chains or chemical moieties of polymers. In preferred embodiments, the gold particles are reduced by heating on a hot plate or in a thermostatic bath and/or by litting by UV-vis or UV lamp, as described in EXAMPLES below. The at least one piece of fibrous material is nonwoven, woven, or any fibrous material known in the art. The method of manufacturing the diagnostic system may further comprise a step of pretreating the at least one piece of fabric in a solution containing a nucleating agent and/or a step of post-treating in a solution containing at least HAuCl₄. In some embodiments, the nucleating agent is ZnO nanoparticles.

### Examples

### Materials for Manufacturing the Gold Particles

Materials used for some embodiments disclosed herein include water dispersion of ZnO nanoparticles (commercial product, for example as obtained from Sigma Aldrich, product number 721077, 20% or 50% wt) where the dispersion is further diluted in distilled water, mother solution of HAuCl₄·3 H₂O (CAS 16961-25-4, PM 393.83 g/mol,) in MeOH with concentration in the range 1% w/w and 0.02 M−15% w/w and 0.35 M, glycine (CAS 56-40-6, PM 75.07 g/mol) solution in distilled water with concentration in the range 0.001 M−2.5 M, isopropanol, 4-(2-hydroxyethyl)-1-piperazineethanesulfonic acid (HEPES, CAS 7365-45-9, PM 238.3012 g/mol) solution in distilled water with concentration in the range 0.001 M−1 M and distilled water.

Pre-Treatment with ZnO Nanoparticles Dispersion

The piece of nonwoven is soaked in ZnO nanoparticles water dispersion (commercial product further diluted in distilled water; from 1 to 60 drops, which means a volume in the range 20 μ-1.2 ml, in 10 ml of water). The piece is dried on paper, then in oven at 100° C. (or higher temperature up to 130° C.) for a time between 15 and 90, e.g. 30, minutes. The process may be repeated more times to enhance the amount of ZnO on the fibers. ZnO nanoparticles may be removed by acidic washing, if needed, after gold nucleation.

ZnO nanoparticles make easier the gold particles synthesis on the fiber, increasing the yield of the next step. Anyway the pre-treatment step is not strictly necessary and it could be skipped.

### Gold Ps are Synthesized In Situ

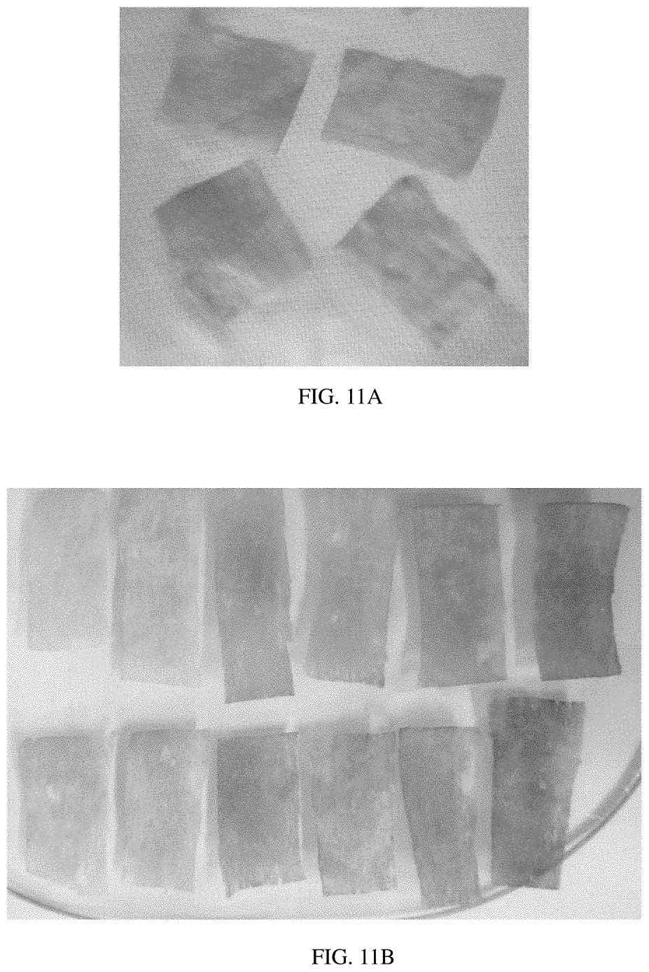

The wanted amount of Au³⁺is added (starting from HAuCl₄ in methanol 1% w/w or more) to a liquid phase (distilled water, isopropanol, distilled water/isopropanol, or a glycine solution). The wanted amount of Au³⁺may be added all in one time at the beginning or splitted in more additions during the synthesis. One or more pieces of nonwoven are put in the liquid phase. The mixture is warmed on hot plate, with or without stirring, and/or lit by UV-Vis lamp (Osram ultra vitalux 300 W) to favour Au reduction; the time range is 1-120 minutes (in some instances, ZnO nanoparticles are added to the nonwoven in a pretreatment step as a nucleating agent, but this pretreatment is not strictly required). Au⁰ grows on the fibers. Gold nucleation on the fibers causes a change of colour. The nonwoven, white at the beginning, can become grey, purple, violet-blue, black, magenta (some examples in FIGS. 11A-B). The final colour depends on dimensions and distribution of the particles on the fibers. The gold particles grow directly on the nonwoven material and they are not linked, embedded, applied or absorbed onto the fibrous material. The gold particles may further grow in a second step, in which the nonwoven material is put again in a liquid phase containing the wanted amount of gold (starting from HAuCl₄ in methanol 1% w/w or more).

One exemplary method of synthesizing gold particles includes the steps of soaking one or more pieces of nonwoven 2 cm×3 cm in ZnO NP water dispersion (2 drops of commercial dispersion in 10 ml of distilled water). The piece is dried on paper, then in oven at 100° C. for 30 minutes. The 0.1 M Glycine solution (2 ml) is diluted in distilled water (6 ml); the pre-treated piece of nonwoven is immersed in such solution. The solution of HAuCl₄·3 H₂O % w/w and 0.02 M in methanol is added (50 μl). The mixture is warmed under stirring to reach the boiling point. The same solution can be used for more pieces of nonwoven, in the same time or in sequence. After 25 minutes the piece of nonwoven, now grey, is taken out from the mixture and it is put on paper towel. It is left to dry at room temperature.

Another exemplary method for synthesing gold particles includes the steps of soaking one or more pieces of nonwoven 2 cm×3 cm in ZnO NP water dispersion (2 drops of commercial dispersion in 10 ml of distilled water). The piece is dried on paper, then in oven at 100° C. for 30 minutes. The pre-treated piece of nonwoven is immersed in a water/isopropanol solution (10 ml of water+5 ml of isopropanol). The solution of HAuCl₄·3 H₂O % w/w and 0.02 M in methanol is added (30 μl). The mixture is warmed to reach the boiling point. The same solution can be used for more pieces of nonwoven, in the same time or in sequence. After 10 minutes the piece of nonwoven, now purple-violet, is taken out from the mixture and it is put on paper towel. It is left to dry at room temperature.

Another exemplary method of synthesizing gold particles includes the steps of soaking one or more pieces of nonwoven 2 cm×3 cm in ZnO NP water dispersion (2 drops of commercial dispersion in 10 ml of distilled water). The piece is dried on paper, then in oven at 100° C. for 30 minutes. The 0.1 M Glycine solution (2 ml) is diluted in distilled water (6 ml). The solution of HAuCl₄·3H₂O 1% w/w and 0.02 M in methanol is added (50 μl). The pre-treated piece of nonwoven is immersed in such solution and it is lit by UV-Vis lamp for a time between 5 and 30 minutes. The same solution can be used for more pieces of nonwoven, in the same time or in sequence. The piece of nonwoven, now grey, is taken out from the mixture and it is put on paper towel. It is left to dry at room temperature.

Another exemplary method for synthesizing gold particles includes the steps of soaking one or more pieces of nonwoven 4 cm×3 cm in ZnO NP water dispersion (6 drops of commercial dispersion in 10 ml of distilled water). The pieces are dried on paper, then in oven at 100° C. for 30 minutes. The solution of HAuCl₄·3 H₂O 7.3% w/w and 0.16 M in methanol is added (15 μl, but even larger volumes) to 50 ml of distilled water; the pre-treated pieces of nonwoven (e.g., up to six) are immersed in the mixture and lit by UV-Vis lamp (Osram ultra vitalux 300 W) for a time between 5 and 90 minutes. The pieces of nonwoven, now purple-violet, are taken out from the mixture, rinsed with distilled water (by immersion) and they are put on paper towel. They are left to dry at room temperature.

Another exemplary method for synthesizing gold particles includes the steps of soaking one or more pieces of nonwoven 4 cm×3 cm in ZnO NP water dispersion (6 drops of commercial dispersion in 10 ml of distilled water). The pieces are dried on paper, then in oven at 100° C. for 30 minutes. The 1 M glycine solution is diluted in distilled water (1.6 ml in 100 ml). The solution of HAuCl₄·3 H₂O 7.3% w/w and 0.16 M in methanol is added (100 μl, but different volumes may be used). The pre-treated pieces of nonwoven (e.g. up to six) are immersed in the mixture and lit by UV-Vis lamp (Osram ultra vitalux 300 W) for a time between 5 and 90 minutes. The pieces of nonwoven, now grey, are taken out from the mixture, rinsed with distilled water (by immersion) and they are put on paper towel. They are left to dry at room temperature.

Another exemplary method for synthesizing gold particles includes the steps of soaking one or more pieces of nonwoven 4 cm×3 cm in ZnO NP water dispersion (6 drops of commercial dispersion in 10 ml of distilled water). The pieces are dried on paper, then in oven at 100° C. for 30 minutes. The 1 M glycine solution is diluted in warm distilled water (1.6 ml in 100 ml), the water was previously warmed on a hot plate to reach a temperature of about 50° C. The solution of HAuCl₄·3H₂O 7.3% w/w and 0.16 M in methanol is added (100 μl, but different volumes may be used). The pre-treated pieces of nonwoven (e.g. up to six) are immersed in the mixture and lit by UV-Vis lamp (Osram ultra vitalux 300 W); the warming is kept during all the exposure, in the range 5-90 minutes. The pieces of nonwoven, now grey, are taken out from the mixture, rinsed with distilled water (by immersion) and they are put on paper towel. They are left to dry at room temperature.

In most preferred method, the synthesis steps include pre-treating the piece of nonwoven by soaking in ZnO nanoparticles water dispersion. The piece is dried on paper, then in oven at 100° C. (or higher temperature up to 130° C.) for 15-90 minutes (it is important to dry the nonwoven, the time depends on the machinery performance). The pre-treated piece of nonwoven is soaked in a water solution containing the wanted amount of Au³⁺(starting from HAuCl₄ in methanol 1% w/w or larger concentration). The same mixture may be used for more pieces of nonwoven, in sequence. The wet nonwoven is lit by UV-Vis lamp (Osram ultra vitalux 300 W) to favour Au reduction and the time of exposure may be 1-2 minutes. This step allows Au⁰ to grow on the fibers, and the nonwoven changes colour, from white to purple-violet or grey. In some cases, the samples may be dried on paper before and/or washed after the UV-vis lamp exposure.

An exemplary protocol of the preferred method includes steps of soaking a piece of nonwoven 3 cm×4 cm in ZnO NP water dispersion (6 drops of commercial dispersion in 10 ml of distilled water). The piece is dried on paper, then in oven at 100° C. for 30 minutes. The diluted gold solution is prepared adding 200 μl of HAuCl₄·H₂O % w/w and 0.02 M in methanol to 4 ml of distilled water. The pre-treated piece of nonwoven is soaked in such solution for about 30 seconds, taken out and dried on paper. The sample is put under the UV-Vis lamp (previously turned on) for 1 minute. The sample, become purple, is soaked in distilled water and then it is put on paper towel, until it is dry.

Another exemplary protocol of the preferred method includes steps of soaking a piece of nonwoven 3 cm×4 cm in ZnO NP water dispersion (6 drops of commercial dispersion in 10 ml of distilled water). The piece is dried on paper, then in oven at 100° C. for 30 minutes. The diluted gold solution is prepared adding 50 μl of HAuCl₄·3H₂O 7.3% w/w and 0.16 M in methanol to a mixture of 3.2 ml of distilled water and 0.8 ml of 1 M glycine. The pre-treated piece of nonwoven is soaked in such solution for about 30 seconds, taken out and dried on paper. The sample is put under the UV-Vis lamp (previously turned on) for 1 minute. The sample, become grey, is soaked in distilled water and then it is put on paper towel, until it is dry.

### Gold Particles Growing Step

After the synthesis step, a further treatment may be used to grow the already nucleated Au Ps. For this purpose in the following examples 4-(2-hydroxyethyl)-1-piperazineethanesulfonic acid (HEPES) is used to favour the gold particles growing, but other compounds could be exploited.

The treatment requires a mixture of a water solution of HEPES (concentration at least 0.001 M) and HAuCl₄·H₂O in methanol (1% w/w or more). Some ml of distilled water may be added to the mixture. Various HEPES/Au³⁺ratios may be used. The HEPES solution may be cooled down in the fridge before use. The liquid phase may be used for the soaking of the whole piece of Au Ps fabric, or for localized growing in drops (drops of the mixture with volume 20-200 μl are put directly on the sample). The growing is achieved through the exposure to UV-Vis or UV lamp, for 5-90 minutes. This step may be even performed by putting the drops on the nonwoven only pre-treated with ZnO NP. After the exposure, the sample is rinsed with distilled water and put on a paper towel, until it is dry.

An exemplary method of growing gold particles starts from the preparation of the liquid phase, mixing distilled water (4 ml), HEPES solution (0.005 M, 6.4 ml) and HAuCl₄ 3H₂O in methanol (1% w/w and 0.02 M, 50 μl, but different volumes may be used). The pre-treated pieces of Au Ps fabric (one or more, even in the same time) are immersed in the mixture and lit by UV-Vis lamp (Osram ultra vitalux 300 W) for a time between 5 and 90 minutes. The pieces of nonwoven, with a more intense color, are taken out from the mixture, rinsed with distilled water (by immersion) and they are put on paper towel. They are left to dry at room temperature.

Another exemplary method of growing gold particles starts from the preparation of the liquid phase, mixing distilled water (4 ml), HEPES solution (0.04 M, 6.4 ml) and HAuCl₄·3 H₂O in methanol (7.3% w/w and 0.16 M, 50 μl, but different volumes may be used). The pre-treated pieces of Au Ps fabric (one or more, even in the same time) are immersed in the mixture and lit by UV-Vis lamp (Osram ultra vitalux 300 W) for a time between 5 and 90 minutes. The pieces of nonwoven, with a more intense color, are taken out from the mixture, rinsed with distilled water (by immersion) and they are put on paper towel. They are left to dry at room temperature.

Another exemplary method of growing gold particles starts from the preparation of the liquid phase, mixing cold HEPES solution (0.005 M, 3 ml) and HAuCl₄·3H₂O in methanol (1% w/w and 0.02 M, 25 μl, but different volumes may be used). Some drops with a volume about 100 μl are put on the pre-treated pieces of Au Ps fabric; the samples are lit by UV-Vis lamp (Osram ultra vitalux 300 W) for a time between 5 and 90 minutes. The pieces of nonwoven, with some spots of more intense color, are rinsed with distilled water (by immersion) and they are put on paper towel. They are left to dry at room temperature.

Another exemplary method of growing gold particles starts from the preparation of the liquid phase, mixing cold HEPES solution (0.04 M, 3 ml) and HAuCl₄·3 H₂O in methanol (7.3% w/w and 0.16 M, 25 μl, but different volumes may be used). Some drops with a volume about 100 μl are put on the pre-treated pieces of Au Ps fabric; the samples are lit by UV-Vis lamp (Osram ultra vitalux 300 W) for a time between 5 and 90 minutes. The pieces of nonwoven, with some spots of more intense color, are rinsed with distilled water (by immersion) and they are put on paper towel. They are left to dry at room temperature.

Another exemplary method of growing gold particles starts from the preparation of the liquid phase, mixing cold HEPES solution (0.04 M, 3 ml) and HAuCl₄·3 H₂O in methanol (7.3% w/w and 0.16 M, 25 μl, but different volumes may be used). Some drops with a volume about 100 μl are put on the pieces of nonwoven treated only with ZnO (pre-treatment step); the samples are lit by UV-Vis lamp (Osram ultra vitalux 300 W) for a time between 5 and 90 minutes. The pieces of nonwoven, with some spots of more intense color, are rinsed with distilled water (by immersion) and they are put on paper towel. They are left to dry at room temperature.

Another exemplary method of growing gold particles starts from the preparation of the liquid phase, mixing cold HEPES solution (0.16 M, 3 ml) and HAuCl₄·3 H₂O in methanol (7.3% w/w and 0.16 M, 100 μl, but different volumes may be used). Some drops with a volume about 100 μl are put on the pre-treated pieces of Au Ps fabric; the samples are lit by UV-Vis lamp (Osram ultra vitalux 300 W) for a time between 5 and 90 minutes. The pieces of nonwoven, with some spots of more intense color, are rinsed with distilled water (by immersion) and they are put on paper towel. They are left to dry at room temperature.

### Characterization of the Produced Samples

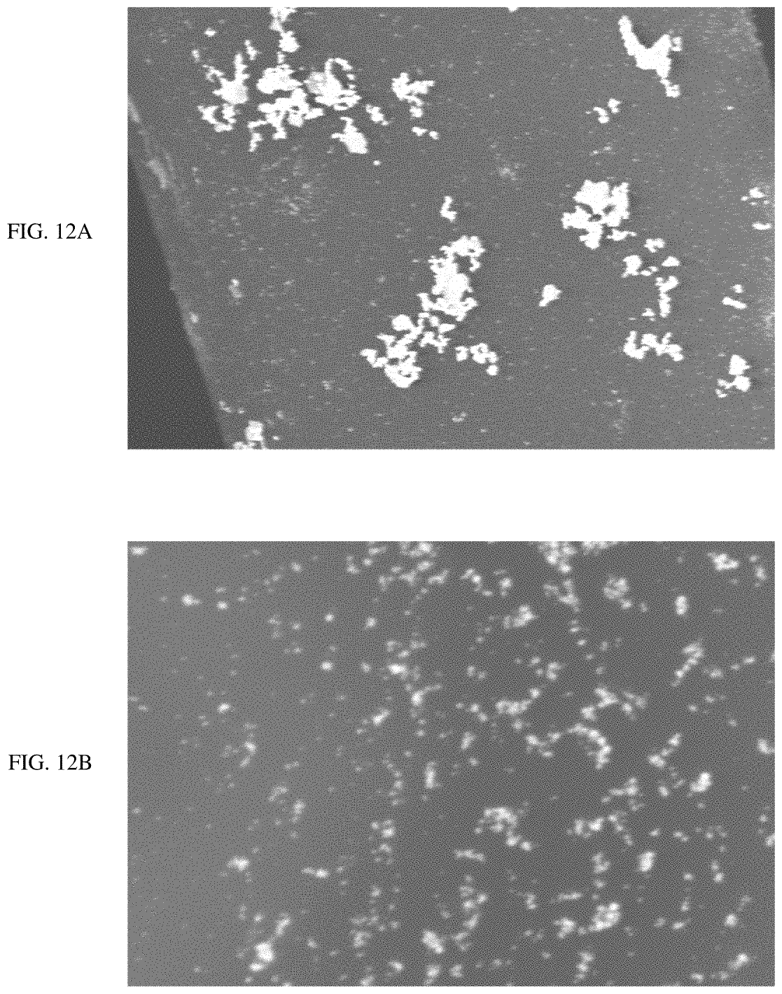

The prepared samples were characterized by using SEM microscopy method where the samples were coated with graphite and the images were taken with in-lens, SE2 and BSD detectors (with the latter, gold particles appear as white dots, as in FIGS. 12A-B).

Reflectance with the integrating sphere was also used at UV-Vis range. The measurements referred to the untreated nonwoven, used as a standard.

FT-IR spectroscopy could also be used to compare the material before and after treatments.

The prepared samples were tested with solutions of N-acetyl Cysteine (NAC, CAS 616-91-1, 163.2 g/mol) in water, to value the interaction between the gold particles on the fibers and a small molecule containing a sulfhydryl group. The interaction was detected mainly by reflectance with the integrating sphere at UV-Vis range.

### Functionalization of Gold Nanoparticles

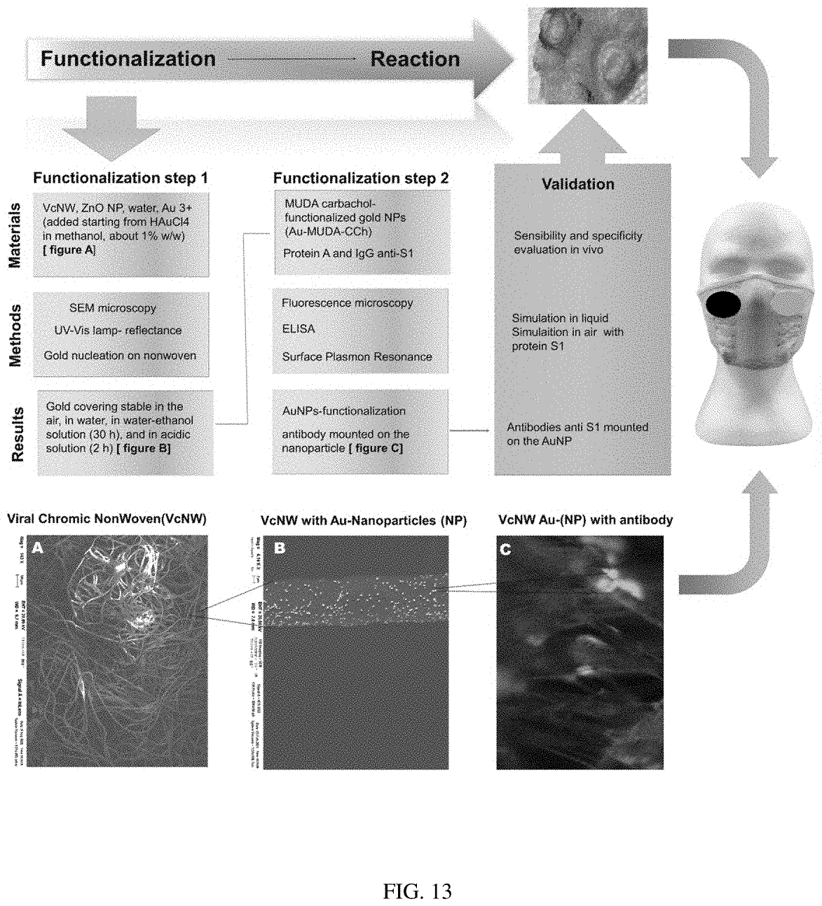

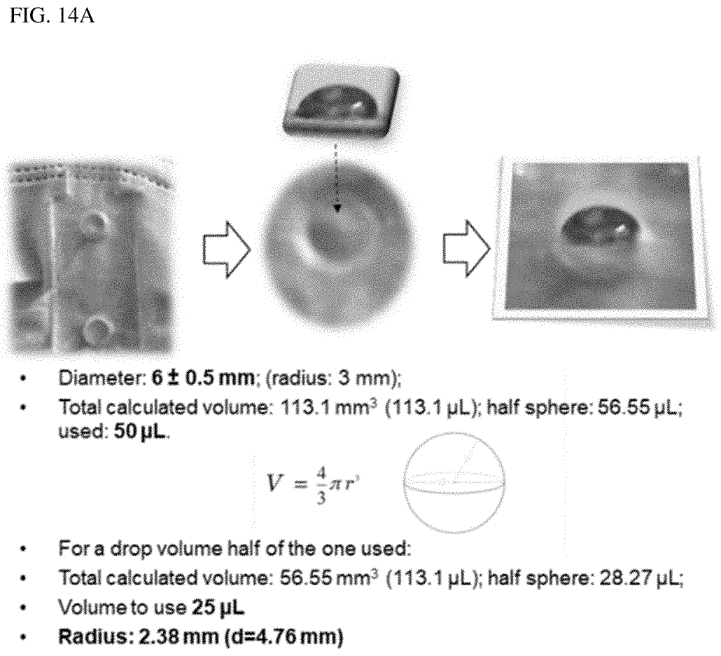

As shown in FIG. 13, two principal steps of functionalization are described herein. The first step describes a direct synthesis of Au-Nanoparticles (Au-NP) onto the nonwoven substrate which results in a final functionalized tissue enriched for Au-NPs. In particular, the gold covering appears stable in the air (even under hairdryer flux), in water, in water-ethanol solution (30 h) and in acidic solution (2 h). The stability was observed in all the tested samples, regardless of the nucleation conditions. The gold release was negligible. The second functionalization step aiming for the assembly of specific antibody on the Au-NPs for assuring the right antigen-antibody reaction. In particular, the protocol used herein assures the binding of the antibody by favouring a conformational arrangement for increasing the efficiency of the antigen-antibody reaction proven combining colorimetric and fluorescent experiments. The fluorescent test confirmed the right antibodies assembly on Au-NPs, and when the antigen is added, also the right molecular reaction antigen-antibody to which corresponding colorimetric change optically appreciable.

The functionalization protocol starts with the step aimed to ensure the binding of the antibody by favouring a conformational arrangement for increasing the efficiency of the antigen-antibody reaction.

The best protocol among those tested in the preliminary trials that gave the best yield is: MUDA carbachol-functionalized gold NPs (Au-MUDA-CCh) with an average diameter of 20-50 nm were synthesized and successively, the incorporation of Protein A and IgG assembled. The protocol individuated assures the right reaction in presence of related antigen. The details of the protocol are reported in TABLE 1.

TABLE 1
chemical steps of gold nanoparticles functionalisation assay

| Step | Chemical | Volume | Concentration | Time |
|---|---|---|---|---|
| Gold chip | MUDA | 1 mL | 10 mM | Overnight |
| functionalisation | | | | |
| EDC/NHS | EDC/NHS | 200 uL | 0.4/0.1 mM | 7 min |
| Protein A | Protein A | 25 uL | 1 mg/mL | 1 hr |
| immobilisation | | | | |
| IgG | IgG | 1:10 | 4.4 mg/mL | 1 hr |

NHS: N-hydroxysuccinimide and
EDC: hydrochloride

### Simulation and Standardization

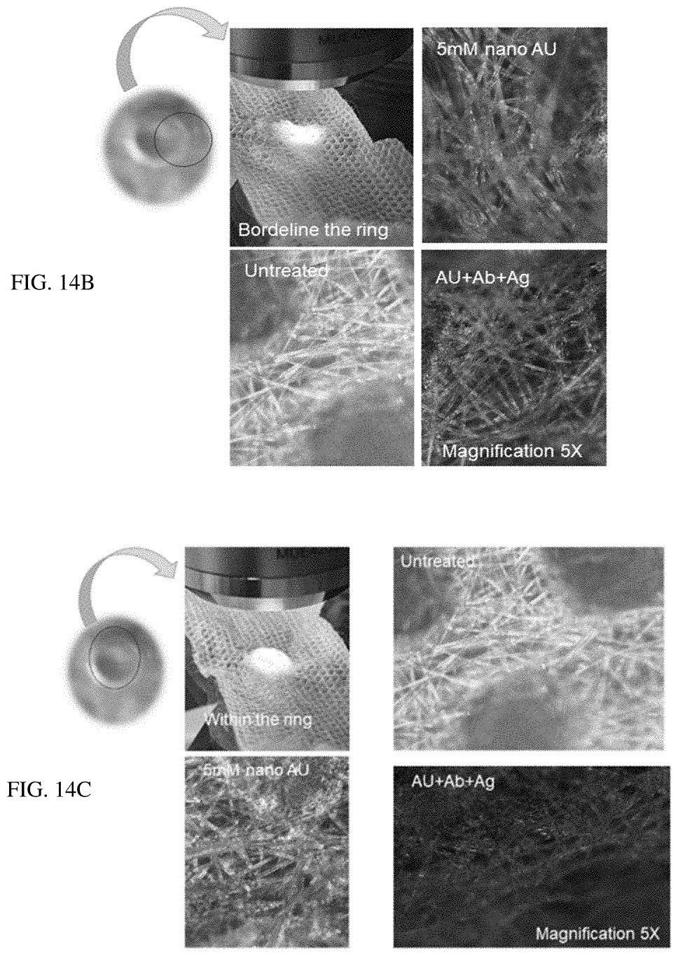

To assess and validate, experiments were performed for optimization of conditions useful to favor the binding kinetic between the free antibody and the nanoparticle-linked antibody with antigen. Standardization tests were carried out on the functionalization process of fabrics conventionally used for the manufacture of masks. FIG. 14A-C show the procedure for identifying the tissue area on which to simulate and standardize functionalization processes with nanoparticles.

### Colorimetric Change Assessment Under Microscopy

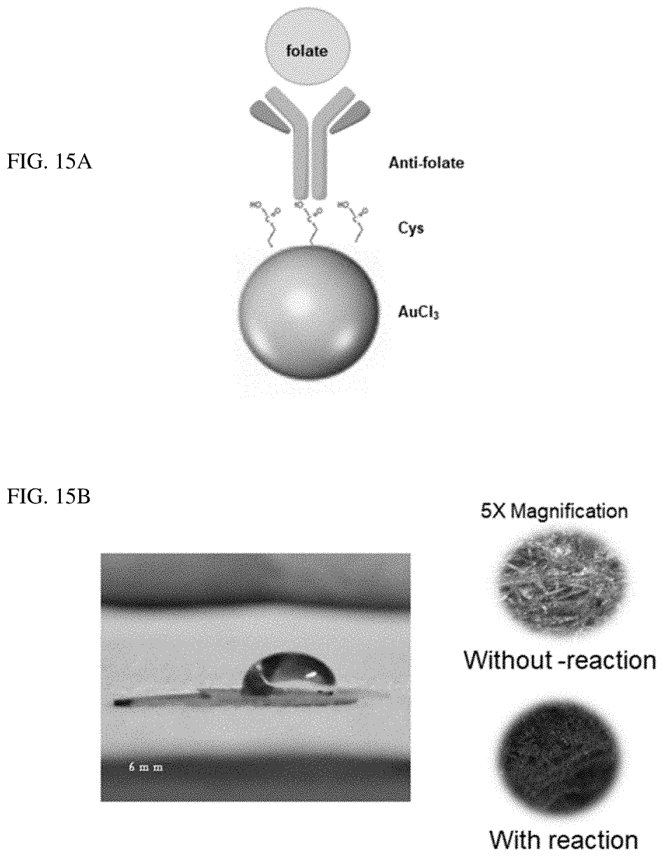

To address the colorimetric change optically appreciable for unit of mask after the antigen-antibody reaction, a series of tests were performed by using a conventional mask-tissue. First, the functionalization process was modified to verify if the replacement of some reagents could modify the colorimetric change under the microscope. FIG. 15A-B and TABLE 2 show the modified protocol and the colorimetric results obtained on an area of diameter 6 mm through 5 mM of gold nanoparticles adsorption.

TABLE 2
chemical steps for modified functionalization protocol

| Step | Chemical | Volume | Concentration | Time |
|---|---|---|---|---|
| Gold | AuCl₃ | 1 mL | 5 mM | 1 hr |
| nanoparticles | | | | |
| Cys | Cystein | 200 uL | 4 mg/mL | 30 min |
| EDC/NHS | EDC/NHS | 200 uL | 0.4/0.1 mM | 7 min |
| IgG anti-folate | IgG | 1:10 | 1 mg/mL | 1 hr |
| Folate | Antigen | 1:10 | 1 mg/mL | 20 min |

### Colorimetric Change Assessment Under Microscopy and Fluorescent Test

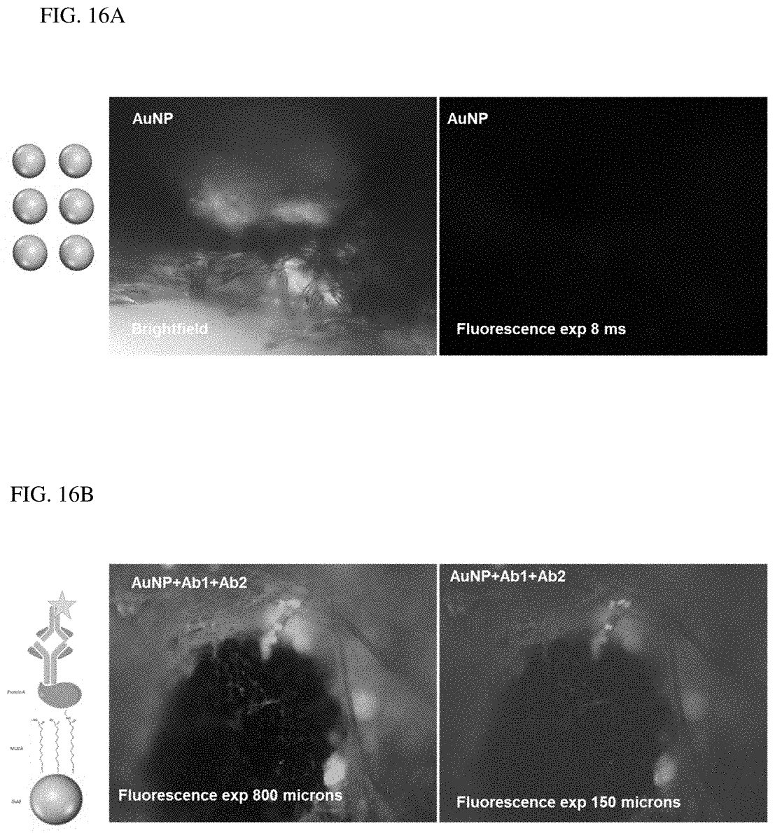

To verify the colorimetric change appreciated under the microscope corresponds to the antigen-antibody reaction, a fluorescent test was carried out. The experiment consisted in using as an antigen a fluorescent antibody capable of recognizing the antibody linked on the gold nanoparticle. After having functionalized the gold nanoparticles adsorbed on the conventional tissue with an antibody, following the protocol and the scheme showed in TABLE 3, a second reaction was carried out by introducing another fluorescent antibody against the first antibody mounted on the nanoparticle. The appreciable colorimetric reaction under the optical microscope (5× magnification). FIGS. 16A-B show the fibres within the 6 mm diameter area. The fluorescent test shows that the colour change appreciable to microscopy corresponds to a successful antigen-antibody reaction.

TABLE 3
chemical steps for fluorescent test

| Step | Chemical | Volume | Concentration | Time |
|---|---|---|---|---|
| Gold | AuCl₃ | 1 mL | 7 mM | 1 hr |
| nanoparticles | | | | |
| MUDA | MUDA | 200 uL | 4 mg/mL | O.N. |
| EDC/NHS | EDC/NHS | 200 uL | 0.4/0.1 mM | 7 min |
| Protein A | Protein | 1:5 | 1 mg/mL | 1 hr |
| IgGrabbit anti | IgG | 1:1 | 1 mg/mL | 1 hr |
| tubulin | | | | |
| Anti-rabbit- | Antigen | 1:1 | 1 mg/mL | 20 min |
| FICHT | | | | |

### Optical Colorimetric Change Assessment on Conventional Mask

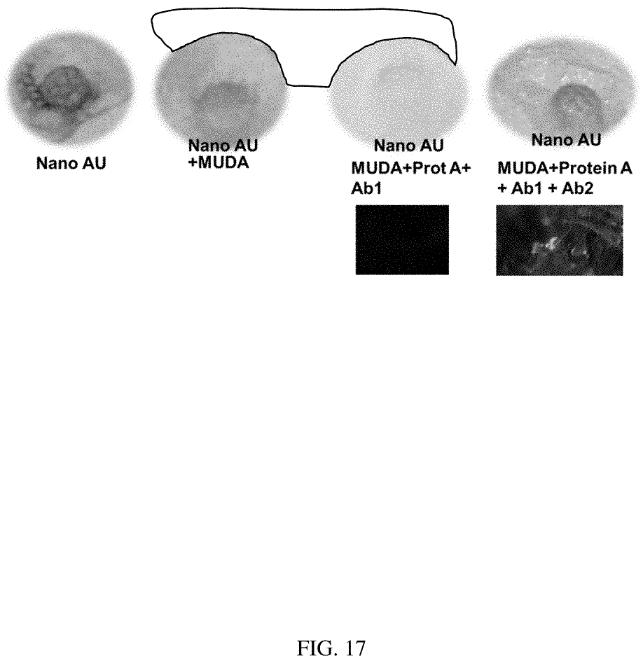

The colorimetric and fluorescent experiment were repeated on a mask and was repeated to evaluate if the colorimetric change was optically appreciable in correspondence to each step of the functionalization protocol. FIG. 17 shows the experimental points of the protocol and the relative modification of the colour. Moreover, the fluorescent test confirmed the molecular reaction at the level of the nanoparticles inserted in the treated fibrous material area.

### Optical Colorimetric Change Assessment on Nonwoven

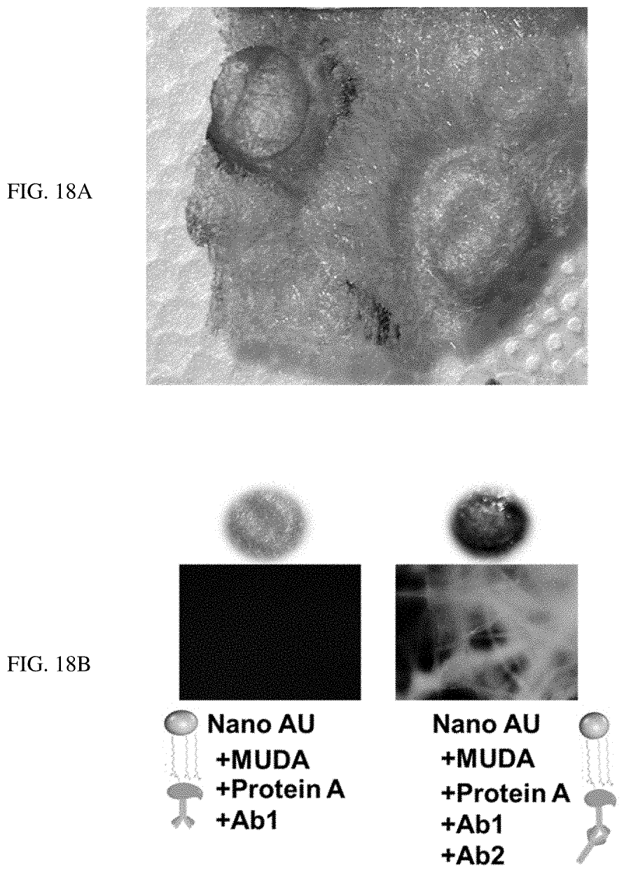

The colorimetric and fluorescent experiment were repeated on nonwoven pre-functionalized with gold nanoparticles (following one of the exemplary method, previously described). FIG. 18A-B show the experimental points of the AuNp-functionalization protocol correspondent to penultimate step, just before the final reaction, and the last step with the complete reaction. The change of colour is evident. The fluorescent test finally confirmed the molecular reaction at the level of the nanoparticles inserted in the treated tissue area.

### Gold Functionalized Nonwoven for Biomedical Applications: An Easy Method for In Situ Nucleation of Au-NPs

Gold nanoparticles (Au-NPs) have intrinsic physicochemical properties suitable for several biomedical applications, such as photothermal therapy, imaging, drug delivery.^{1,2,3} When Au-NPs are immobilized on surfaces, it is possible to obtain new nano-based smart materials that allow to exploit Au-NPs properties in an easier to handle form. For this aim, many methods to apply Au-NPs to fibrous materials have been already described, both with previously prepared nanoparticles and with in situ gold reduction;⁴ however, these preparations are often expansive, time consuming, and feasible on a laboratory scale only. In the present work, we propose a fast and simple method for the direct functionalization of polymeric nonwoven with Au-NPs. The synthesis just requires a gold precursor, a nucleating agent and UV-Vis light. Our protocol is based on previously reported methods for gold deposition on other surfaces, such as diatomaceous earth⁵ or ZnO tetrapods.⁶

In the first step, the textile is pre-treated with the nucleating agent, mainly titanium or zinc oxide nanoparticles (TiO₂—NPs, ZnO-NPs) in water dispersion. In the second step, the sample is immersed in a HAuCl₄·3H₂O aqueous solution (with or without few ml of isopropanol), and it is put under a halogen lamp. The gold nucleation occurs in 20-30 minutes directly on the fibers and the covering degree can be modified just changing the few experimental parameters (i.e. gold concentration, nucleating agent's amount, time of exposure).

The samples were characterized by SEM microscopy: the Au-NPs appear as white dots on the fibers, and their distribution depends on the synthesis conditions. The gold nucleation was confirmed by reflectance analyses: the treated samples show a minimum in the range 530-600 nm, compatible with the gold plasmonic absorption.

The Au-NPs enriched nonwoven may be a platform for further functionalizations, for example with enzymes or drugs,^{1,3} that may enable the development of functional nanomaterials for biomedical applications. Moreover, a possible synergy between Au-NPs and ZnO-NPs or TiO₂—NPs, used here as nucleating agents, could advantageously enhance the physicochemical properties of the nonwoven fabric. ZnO—NPs and TiO₂—NPs, indeed, have well-known photocatalytic properties and they are widely used to produce nano-improved textiles.⁷

### REFERENCES

¹ Aminabad et al., Cell Biochemistry and Biophysics 2019, 77, 123-137
² Zhang et al., Chem. Sci. 2020, 11, 923-936
³ Hu et al., Front. Bioeng. Biotechnol. 2020, 8, 990
⁴ Mehravani et al., Nanomaterials 2021, 11, 1067
⁵ Villani et al., Micro and Nano Engineering 2019, 2, 29-34
⁶ Bertoni et al., Sci. Rep. 2016, 6, 19168
⁷ Krifa et al., J. Text. Inst. 2020, 1721696

It is to be understood that this invention is not limited to any particular embodiment described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

We claim:

1. A diagnostic system, comprising:
at least one layer of fibrous material;
a color changing moiety positioned on or within the at least one layer of fibrous material, wherein the color changing moiety is comprised of
gold particles,
at least one linker linked to the gold particles,
an anchor protein linked to the at least one linker, and
at least one antibody or antibody fragment conjugated to the anchor protein,
wherein the at least one antibody or antibody fragment has an affinity for an antigen of interest, and
wherein the color changing moiety changes color when the at least one antibody or antibody fragment binds to the antigen of interest.

2. The diagnostic system of claim 1, wherein the gold particles are gold nanoparticles.

3. The diagnostic system of claim 1, wherein the at least one linker has a sulfhydryl moiety and a carboxylic acid moiety.

4. The diagnostic system of claim 3, wherein the at least one linker is 11-Mercaptoundecanoic acid (MUDA) or cysteine (Cys).

5. The diagnostic system of claim 1, wherein the anchor protein is selected from protein A, protein A ZZ domain, protein G, protein L, and fragments thereof.

6. The diagnostic system of claim 1, wherein the at least one layer of fibrous material is nonwoven.

7. The diagnostic system of claim 1, wherein the at least one layer of fibrous material is woven.

8. The diagnostic system of claim 1, wherein the antigen of interest is a protein having a molecular weight of about 45 to 250 kDa.

9. The diagnostic system of claim 1, wherein the antigen of interest is SARS-CoV-2 virus S protein.

10. The diagnostic system of claim 1, wherein the antigen of interest is receptor of advanced glycation end products (RAGE).

11. The diagnostic system of claim 1, wherein the gold particles are immobilized on or bound to one or more fibers of the at least one layer of fibrous material.

12. The diagnostic system of claim 1, wherein the diagnostic system is configured as a face mask wearable by a human or non-human animal subject.

13. The diagnostic system of claim 1, wherein the diagnostic system is configured as a garment.

14. The diagnostic system of claim 1, wherein the diagnostic system is configured as a bedding cover.

15. The diagnostic system of claim 1, wherein the diagnostic system is configured as a wipe.

16. The diagnostic system of claim 15, wherein the wipe is configured for use on animal skin.

17. The diagnostic system of claim 15, wherein the wipe is configured for use on substrates other than animal skin.

18. The diagnostic system of claim 1, wherein the diagnostic system is configured as a surface cover.

19. A method for detecting one or more antigens, comprising:
bringing a fluid or surface which is contaminated with the one or more antigens into contact with the diagnostic system of claim 1; and
detecting a change in color on at least one portion of the diagnostic system when the one or more antigens become bound to the at least one antibody or antibody fragment capable of recognizing the one or more antigens.

20. The method of claim 19, wherein the detection is made by optical observation.

21. The method of claim 19, wherein the optical observation occurs within about 10-30 minutes of binding of the one or more antigens to the at least one antibody or antibody fragment capable of recognizing the one or more antigens.

22. The method of claim 19, further comprising a step of assessing an amount of coupling between the antigens to antibody or antibody fragment capable of recognizing the one or more antigens.

23. The method of claim 22, wherein the amount of coupling is calculated by comparing a degree of colorimetric change after contact with the fluid or surface with a known degree of colorimetric change after contact with a known amount of an antigen of interest.

24. The method of claim 19, wherein the antigen of interest is a protein having a molecular weight of about 45 to 250 kDa.

25. The method of claim 19, wherein the one or more antigens comprise SARS-CoV-2 virus S protein.

26. The method of claim 19, wherein the one or more antigens comprise receptor of advanced glycation end products (RAGE).

27. The method of claim 19, wherein the fluid or surface brought into contact with the diagnostic system is or comprises breath exhaled from a human or non-human animal subject.

28. The method of claim 19, wherein the fluid or surface is or comprises a bodily discharge of a subject.

29. The method of claim 19, wherein the fluid of interest does not travel across or through a surface of the at least one layer of fibrous material.

30. A method of manufacturing the diagnostic system of claim 1, comprising the steps of:
synthesizing gold particles (Au Ps) on at least one piece of fabric by
adding the at least one piece of fabric to a HAuCl₄ solution to nucleate Au⁰ directly on the fabric to form Au Ps fabric,
conjugating at least one linker to the Au Ps fabric to form linker-coupled gold particles fabric;
linking at least one anchoring protein to the linker; and
conjugating at least one antibody or antibody fragment capable of recognizing the one or more antigens to the at least one anchoring protein.

31. The method of claim 30, wherein the gold particles are gold nanoparticles.

32. The method of claim 30, wherein the at least one linker has a sulfhydryl moiety and a carboxylic acid moiety.

33. The method of claim 32, wherein the at least one linker is 11-Mercaptoundecanoic acid (MUDA) or cysteine (Cys).

34. The method of claim 30, wherein the at least one piece of fabric is nonwoven.

35. The method of claim 30, wherein the at least one piece of fabric is woven.

36. The method of claim 30, wherein the HAuCl₄ solution comprises a reducing agent.

37. The method of claim 30, further comprising pretreating the at least one piece of fabric in a solution containing a nucleating agent.

38. The method of claim 37, wherein the nucleating agent is ZnO nanoparticles (ZnO NPs).

39. The method of claim 30, wherein the at least one anchoring protein is selected from protein A, protein A ZZ domain, protein G, protein L, and fragments thereof.

40. The method of claim 30, further comprising adding the Au Ps fabric to a HAuCl₄ solution to further grow nucleated Au Ps.
